(19) United States
(12) Patent Application Publication
(10) Pub. No.: US 2022/0348901 A1
(43) Pub. Date: Nov. 3, 2022

(54) METHODS, DEVICES, AND KITS FOR PURIFYING AND LYSING BIOLOGICAL PARTICLES

(71) Applicant: 10x Genomics, Inc., Pleasanton, CA (US)

(72) Inventors: Stephane Claude BOUTET, Pleasanton, CA (US); Alireza SALMANZADEH, San Francisco, CA (US); Michael GIBBONS, Pleasanton, CA (US)

(21) Appl. No.: 17/551,761

(22) Filed: Dec. 15, 2021

Related U.S. Application Data
(60) Provisional application No. 63/224,164, filed on Jul. 21, 2021.
(60) Provisional application No. 63/125,626, filed on Dec. 15, 2020.

(51) Int. Cl. C12N 15/10; B01L 3/00

(52) U.S. Cl. CPC C12N 15/1017; B01L 3/5021; B01L 3/502753; B01L 3/502761; B01L 2300/042; B01L 2300/0681; B01L 2300/0832; B01L 2400/0683

(57) ABSTRACT

Devices, kits, and their methods of use for lysing and/or purifying biological particles, e.g., nuclei are provided. One or more thixotropic layers can be employed in a vessel to purify biological particles. A device with sharp features may be employed to lyse biological particles or the contents thereof.

### BACKGROUND OF THE INVENTION

Many biomedical applications rely on high-throughput assays of samples combined with one or more reagents in droplets or particles. For example, in both research and clinical applications, high-throughput genetic tests using target-specific reagents are able to provide information about samples in drug discovery, biomarker discovery, and clinical diagnostics, among others. Many of these applications rely on the presence of a biological particle (e.g., a cell or particulate component thereof, e.g., a nucleus). However, precise sample preparation may be required before droplet formation. Prior devices and methods for purifying biological particles may disturb and alter its characteristics (e.g., gene expression, activation, or viability). Accordingly, devices and methods for gently and reliably lysing and purifying biological particles would be beneficial.

### SUMMARY OF THE INVENTION

In one aspect, the invention features a method of purifying a biological particle. The method includes providing a vessel that includes an inlet; a first layer including a first liquid having a first density; and a thixotropic layer disposed above the first layer. The method further includes providing a liquid mixture that includes the biological particle to the inlet of the vessel, wherein the density of the liquid mixture is less than the first density; and centrifuging the vessel. The thixotropic layer separates the first layer and the liquid mixture and allows passage of the biological particle to the first layer during centrifugation.

In some embodiments, the method further includes removing a supernatant liquid after centrifugation.

In some embodiments, the vessel is inserted in an outer vessel.

In some embodiments, the vessel includes an outlet.

In some embodiments, the method further includes opening a tip of the vessel to create the outlet.

In some embodiments, the method further includes centrifuging the vessel, wherein the biological particle exits the vessel via the outlet and is collected in the outer vessel.

In some embodiments, the first layer and/or the liquid mixture includes iodixanol.

In some embodiments, the first layer includes about 30% iodixanol.

In some embodiments, the liquid mixture includes about 25% iodixanol.

In some embodiments, the thixotropic layer includes a polymeric gel.

In some embodiments, the polymeric gel includes polyester.

In some embodiments, the biological particle is a cell or a particulate component thereof.

In some embodiments, the biological particle is an organelle, e.g., a nucleus.

In another aspect, the invention features a device that includes a centrifuge tube containing a liquid having a predetermined density that is composed of tissue culture medium. For example, the device may contain a liquid having tissue culture medium and a concentration of from about 10% to about 50% iodixanol (e.g., from about 15% to about 45%, from about 20% to about 35%, from about 22% to about 32%, or from about 24% to about 30%).

The centrifuge tube may be, for example, a microcentrifuge tube or a PCR tube, or any other suitable structure configured to fit within a centrifuge.

In some embodiments, the density of the liquid is from about 1.11 g/mL to about 1.12 g/mL (e.g., from about 1.146 g/mL to about 1.175, e.g., about 1.1107 g/mL, 1.117 g/mL, 1.127 g/mL, 1.136 g/mL, 1.146 g/mL, 1.156 g/mL, 1.165 g/mL, or 1.175 g/mL). The liquid may contain iodixanol or a comparable component to create a similar density as iodixanol, such as ficoll, histopaque, or sucrose.

In another aspect, the invention features a method of purifying a biological particle by providing a device that includes the centrifuge tube. The method further includes providing a liquid mixture that includes the biological particle to the centrifuge tube and centrifuging the centrifuge tube. During centrifugation, the biological particle moves to the bottom of the centrifuge tube.

In one embodiment, a first centrifugation step is performed in which all contents of the tissue culture medium are pelleted. Following the first centrifugation, the supernatant may be removed, e.g., via decanting or discarding. A cell culture medium may then be added to the pellet. When this sample is centrifuged, only certain (e.g., desired) biological particles (e.g., cells or particulate components thereof, e.g., nuclei) are pelleted.

In another aspect, the invention features a kit that includes an outer vessel and a removable insert configured to fit in the vessel. The removable insert includes an inlet; a first layer including a first liquid having a first density; and a thixotropic layer disposed above the first layer. The thixotropic layer is configured to separate the first layer and a diluent having a density lower than the first density provided to the inlet of the insert.

In some embodiments, the removable insert includes an outlet. The outlet may include a breakable or removable tip.

In some embodiments, the first layer includes iodixanol.

In some embodiments, the first layer includes about 30% iodixanol.

In some embodiments, the kit further includes the diluent. The diluent may include about 25% iodixanol.

In some embodiments, the thixotropic layer includes a polymeric gel. The polymeric gel may include polyester.

In some embodiments, the kit further includes a cell lysis buffer. The cell lysis buffer may include an RNAse inhibitor. The cell lysis buffer may include a surfactant (e.g., from about 0.01% to about 1.0% of the surfactant). In some embodiments, the cell lysis buffer includes 10 mM Tris-HCl (pH 7.4), 10 mM NaCl, 3 mM MgCl₂, 0.01% IGEPAL CA-630 (octylphenoxypolyethoxyethanol), 0.2 U/μl RNase Inhibitor (e.g., enzymatic RNAse inhibitor, e.g., Protector RNAse), 0.1% BSA, 2 mM spermine, and from about 0.01% to about 1.0% of a surfactant/detergent (e.g., TWEEN-20 (polysorbate 20) or IGEPAL CA-630 (octylphenoxypolyethoxyethanol). For example, the cell lysis buffer may include 10 mM Tris-HCl (pH 7.4), 10 mM NaCl, 3 mM MgCl₂, 0.01% IGEPAL CA-630 (octylphenoxypolyethoxyethanol), 0.2 U/μl SUPERase In RNase Inhibitor, 0.1% BSA, 2 mM spermine, and 0.1% TWEEN-20 (polysorbate 20).

In some embodiments, the kit further includes an isotonic buffer. The isotonic buffer may include an RNAse inhibitor. The isotonic buffer may include a surfactant (e.g., from about 0.01% to about 1.0% of the surfactant). The isotonic buffer may include 20 mM Tris-HCl (pH 7.4), 0.25 M sucrose, 25 mM KCl, 5 mM MgCl₂, 0.01% IGEPAL CA-630, 0.2 U/μl SUPERase In RNase Inhibitor, 0.1% BSA, 2 mM spermine, and 0.1% TWEEN-20 (polysorbate 20).

In some embodiments, the kit further includes a cap configured to seal the inlet of the removable insert.

In another aspect, the invention features a device for lysing biological particles that includes a flow path having an inlet; an outlet; and at least one feature with a corner radius of less than about 1 mm disposed in the flow path.

In some embodiments, the flow path includes a plurality of features, each feature having a corner radius of less than about 1 mm and disposed in the flow path.

In some embodiments, at least two of the features are positioned in register and on opposite sides of the flow path.

In some embodiments, the plurality of features are arranged in a sawtooth pattern.

In some embodiments, the device includes a plurality of flow paths, each flow path having an inlet; an outlet; and at least one feature with a corner radius of less than about 1 mm disposed in the flow path.

In some embodiments, the plurality of flow paths are substantially parallel.

In some embodiments, the device further includes a filter positioned in the flow path at or upstream of the outlet and configured to trap particles of a predetermined size.

In another embodiment, the invention features a kit that includes a vessel and a device for lysing biological particles that includes a flow path having an inlet; an outlet; and at least one feature with a corner radius of less than about 1 mm disposed in the flow path. The device is configured to fit in the vessel.

In some embodiments, the kit further includes a cap configured to seal the inlet of the device.

In some embodiments, the flow path includes a plurality of features, each feature having a corner radius of less than about 1 mm and disposed in the flow path.

In some embodiments, at least two of the features are positioned in register and on opposite sides of the flow path.

In some embodiments, the plurality of features are arranged in a sawtooth pattern.

In some embodiments, the device includes a plurality of flow paths, each flow path having an inlet; an outlet; and at least one feature with a corner radius of less than about 1 mm disposed in the flow path.

In some embodiments, the plurality of flow paths are substantially parallel.

In some embodiments, the kit further includes a filter positioned in the flow path at or upstream of the outlet and configured to trap particles of a predetermined size.

In another aspect, the invention features a method for lysing biological particles. The method includes providing a device including a flow path having an inlet and an outlet, the flow path having at least one feature with a corner radius of less than about 1 mm and disposed in the flow path. The method further includes flowing a liquid mixture including the biological particles through the flow path. The at least one feature lyses the biological particles, and at least a portion of the biological particles or the contents thereof exit the flow path via the outlet.

In some embodiments, the method further includes washing the biological particles in the flow path.

In some embodiments, the method further includes providing magnetic particles that bind to at least a portion of the biological particles or the contents thereof.

In another aspect, the invention features a method for lysing biological particles. The method includes providing a vessel and a device having a flow path with an inlet and an outlet, the flow path including at least one feature with a corner radius of less than about 1 mm and disposed in the flow path, wherein the device is positioned in the vessel. The method further includes providing a liquid mixture including the biological particles to the inlet of the device and centrifuging the device in the vessel. Upon centrifugation, the at least one feature lyses the biological particles, and at least a portion of the biological particles or the contents thereof exit the flow path via the outlet and are collected in the vessel.

In another aspect, the invention features a kit that includes a vessel and a device being configured to fit in the vessel. The device includes an inlet; an outlet; and a filter positioned in the device at or upstream of the outlet and configured to trap particles of a predetermined size.

In some embodiments, the device further includes a layer that includes a flow path with at least one feature with a corner radius of less than about 1 mm and disposed in the flow path. The layer is disposed at or upstream of the outlet (e.g., upstream or downstream of the filter).

In some embodiments, the layer includes a plurality of flow paths, each flow path in the layer having an inlet; an outlet; and at least one feature with a corner radius of less than about 1 mm disposed in the flow path.

In some embodiments, the plurality of flow paths in the layer are substantially parallel.

In some embodiments, the vessel includes a medium having a density of greater than 1.0 g/m.

In another aspect, the invention features a method for purifying a second subset of biological particles using a kit as described herein. The method includes providing the kit, providing a liquid mixture with biological particles to the inlet of the device; and centrifuging the device in the vessel. A first subset of the biological particles is trapped by the filter, and a second subset of the biological particles exits the outlet and is collected in the vessel.

In some embodiments, the vessel includes a medium having a density of greater than 1.0 g/m, and the second subset of biological particles is in the medium, e.g., pelleted during centrifugation.

In another aspect, the invention features a method for lysing biological particles using a kit as described herein that includes a device (e.g., having a layer with a flow path having at least one feature with a corner radius of less than about 1 mm disposed in the flow path) and a vessel to house the device. The method includes providing the kit, providing a liquid mixture with biological particles to the inlet of the device; and centrifuging the device in the vessel. The at least one feature lyses the biological particles, and at least a portion of the contents of the biological particles exits the flow path via the outlet and is collected in the vessel.

In another aspect, the invention features a kit for lysing biological particles. The kit includes a vessel that includes a plurality of features having a corner radius of less than about 1 mm disposed along a wall of the vessel. The kit further includes a pestle configured to fit within the vessel. The pestle may include a plurality of features having a corner radius of less than about 1 mm disposed along a wall of the pestle.

In some embodiments, the plurality of features of the vessel and/or the plurality of features of the pestle is arranged in a sawtooth pattern.

In another aspect, the invention features a method of lysing biological particles. The method includes providing a kit of as described herein; providing a liquid mixture including the biological particles in the vessel; and rotating the pestle in the vessel. The plurality of features of the vessel and/or the plurality of features of the pestle lyse the biological particles.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “about,” as used herein, refers to ±10% of a recited value.

The terms “adaptor(s),” “adapter(s),” and “tag(s)” may be used synonymously. An adaptor or tag can be coupled to a polynucleotide sequence to be “tagged” by any approach including ligation, hybridization, or other approaches.

The term “barcode,” as used herein, generally refers to a label, or identifier, that conveys or is capable of conveying information about an analyte. A barcode can be part of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads in real time.

The term “support,” as used herein, generally refers to a particle that is not a biological particle. The particle may be a solid or semi-solid particle. The particle may be a bead, such as a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “biological particle,” as used herein, generally refers to a discrete biological system derived from a biological sample. The biological particle may be a virus. The biological particle may be a cell or derivative of a cell. The biological particle may be an organelle from a cell. Examples of an organelle from a cell include, without limitation, a nucleus, endoplasmic reticulum, a ribosome, a Golgi apparatus, an endoplasmic reticulum, a chloroplast, an endocytic vesicle, an exocytic vesicle, a vacuole, and a lysosome. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or another organelle of a cell. A cell may be a live cell. The live cell may be capable of being cultured, for example, being cultured when enclosed in a gel or polymer matrix, or cultured when comprising a gel or polymer matrix.

The term “fluidically connected,” as used herein, refers to a direct connection between at least two device elements, e.g., a channel, reservoir, etc., that allows for fluid to move between such device elements without passing through an intervening element.

The term “genome,” as used herein, generally refers to genomic information from a subject, which may be, for example, at least a portion or an entirety of a subject's hereditary information. A genome can be encoded either in DNA or in RNA. A genome can comprise coding regions that code for proteins as well as non-coding regions. A genome can include the sequence of all chromosomes together in an organism. For example, the human genome has a total of 46 chromosomes. The sequence of all of these together may constitute a human genome.

The term “in fluid communication with,” as used herein, refers to a connection between at least two device elements, e.g., a channel, reservoir, etc., that allows for fluid to move between such device elements with or without passing through one or more intervening device elements.

The term “in register,” as used herein, refers to a pair of sharp features that are substantially aligned. For example, a pair of sharp features that are in register may have the tips of the sharp features substantially aligned on opposite sides of a flow path, e.g., the tips may be offset by no more than 50 μm, e.g., no more than 40, 30, 20, 10, 5, or 1 μm. A device with a plurality of sharp features positioned on opposite sides of the flow path may have multiple matched pairs where the tips of the sharp features of each matched pair are substantially aligned on opposite sides of the flow path.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA or a DNA molecule. The macromolecular constituent may comprise RNA or an RNA molecule. The RNA may be coding or non-coding. The RNA may be messenger RNA (mRNA), ribosomal RNA (rRNA) or transfer RNA (tRNA), for example. The RNA may be a transcript. The RNA molecule may be (i) a clustered regularly interspaced short palindromic (CRISPR) RNA molecule (crRNA) or (ii) a single guide RNA (sgRNA) molecule. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide or a protein. The polypeptide or protein may be an extracellular or an intracellular polypeptide or protein. The macromolecular constituent may also comprise a metabolite. These and other suitable macromolecular constituents (also referred to as analytes) will be appreciated by those skilled in the art (see U.S. Pat. Nos. 10,011,872 and 10,323,278, and PCT Publication No. WO 2019/157529, each of which is incorporated herein by reference in its entirety).

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise an oligonucleotide or polypeptide sequence. The molecular tag may comprise a DNA aptamer. The molecular tag may be or comprise a primer. The molecular tag may be or comprise a protein. The molecular tag may comprise a polypeptide. The molecular tag may be a barcode.

The term “oil,” as used herein, generally refers to a liquid that is not miscible with water. An oil may have a density higher or lower than water and/or a viscosity higher or lower than water.

The term “particulate component of a cell” refers to a discrete biological system derived from a cell or fragment thereof and having at least one dimension of 0.01 μm (e.g., at least 0.01 μm, at least 0.1 μm, at least 1 μm, at least 10 μm, or at least 100 μm). A particulate component of a cell may be, for example, an organelle, such as a nucleus, an exosome, a liposome, an endoplasmic reticulum (e.g., rough or smooth), a ribosome, a Golgi apparatus, a chloroplast, an endocytic vesicle, an exocytic vesicle, a vacuole, a lysosome, or a mitochondrion.

The term “sample,” as used herein, generally refers to a biological sample of a subject. The biological sample may be a nucleic acid sample or protein sample. The biological sample may be derived from another sample. The sample may be a tissue sample, such as a biopsy, core biopsy, needle aspirate, or fine needle aspirate. The sample may be a liquid sample, such as a blood sample, urine sample, or saliva sample. The sample may be a skin sample. The sample may be a cheek swap. The sample may be a plasma or serum sample. The sample may include a biological particle, e.g., a cell or virus, or a population thereof, or it may alternatively be free of biological particles. A cell-free sample may include polynucleotides. Polynucleotides may be isolated from a bodily sample that may be selected from the group consisting of blood, plasma, serum, urine, saliva, mucosal excretions, sputum, stool and tears.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by ILLUMINA®, Pacific Biosciences (PACBIO®), Oxford NANOPORE®, or Life Technologies (ION TORRENT®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “subject,” as used herein, generally refers to an animal, such as a mammal (e.g., human) or avian (e.g., bird), or other organism, such as a plant. The subject can be a vertebrate, a mammal, a mouse, a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, or an individual that is in need of therapy or suspected of needing therapy. A subject can be a patient.

The term “substantially stationary,” as used herein with respect to droplet or particle formation, generally refers to a state when motion of formed droplets or particles in the continuous phase is passive, e.g., resulting from the difference in density between the dispersed phase and the continuous phase.

### BRIEF DESCRIPTION OF THE DRAWINGS

FIG. 1 is a schematic diagram of a kit of the invention. The kit includes an outer vessel and a removable insert configured to fit in the vessel. The removable insert includes an inlet and a thixotropic layer that separates liquid layers with different densities.

FIG. 2 is a schematic diagram of a method as described herein. A vessel containing a liquid mixture with a suspension of biological particles and debris is centrifuged. The supernatant liquid is discarded, and the biological particles are resuspended in a new liquid without debris.

FIG. 3 is a schematic diagram of a device as described herein. The device includes a plurality of flow paths containing sharp features configured to lyse a cell as it passes through the flow path. Upon lysis, the contents of the cell, such as cytoplasmic RNA and nuclei, are released from the cell.

FIG. 4 is a schematic diagram of a device as in FIG. 3 and further includes a filter configured to trap particles of a predetermined size.

FIG. 5 is a schematic diagram of a device as in FIG. 3 that further includes a plurality of magnetic particles configured to bind to and trap certain biological material, such as cytoplasmic RNA.

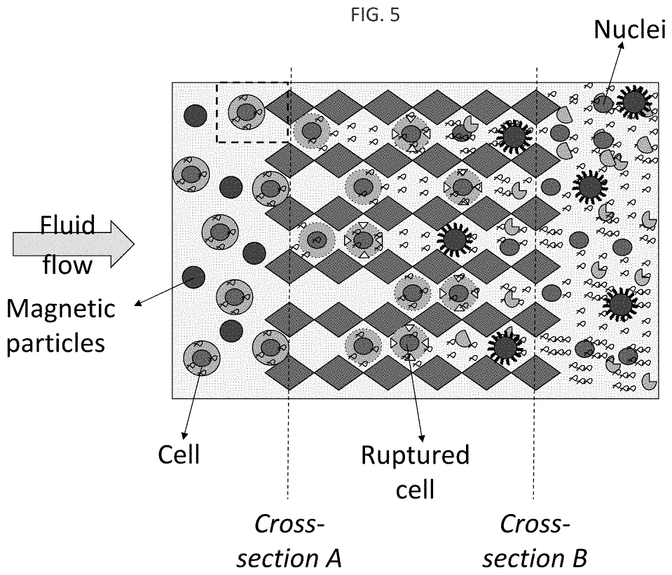

FIGS. 6A and 6B are schematic diagrams of cross-sections of a device and correspond to cross-section A and cross-section B as shown in FIGS. 3 and 5. FIG. 6A shows intact cells, and FIG. 6B shows lysed cells.

FIG. 7 is a schematic diagram showing a kit that includes a device including a plurality of flow paths containing sharp features configured to lyse a cell as it passes through the flow path. The device is configured to fit within a vessel and contains a cap that seals the inlet of the device. Upon centrifugation, the cells are ruptured, and nuclei collect at the bottom of the vessel.

FIG. 8 is a schematic diagram showing a closeup view of the device in the kit of FIG. 7.

FIG. 9 is a schematic drawing showing a kit and method as described herein. The kit includes a device and a vessel and is configured to collect biological particles. The device contains a polymer filter and a vessel containing a dense media that collects biological particles. Shown on the left is the vessel with a suspension of biological particles. The kit can then be centrifuged, vortexed, and centrifuged again to collect a pellet of the biological particles as shown on the right.

FIG. 10 is a schematic drawing showing a kit as described herein containing a device that fits within a vessel. The device includes a layer of textured channels (e.g., containing sharp features) configured to lyse the biological particles. The device further includes a filter layer (e.g., porous filter) and an outlet to allow the contents of the biological particles (e.g., nuclei) to collect in the vessel. The vessel further includes a dense media to collect the biological particles.

FIG. 11 is a schematic drawing showing a kit as described herein. The kit contains a textured pestle and a textured lysis column (e.g., containing sharp features). The pestle fits within the column and can be rotated to lyse biological particles.

### DETAILED DESCRIPTION OF THE INVENTION

The invention provides devices and kits for purifying or lysing biological particles (e.g., cells or particulate components thereof, e.g., nuclei) and methods of their use. The methods, devices, and kits may be used to purify biological particles of a desired property and/or size. The purified biological particles may then be suitable for incorporation into droplets or particles that may be used as microscale chemical reactors, e.g., for genetic sequencing. In general, the kits described herein include a vessel that includes an inlet and a thixotropic layer disposed in the vessel. The vessel may further include an outlet to allow flow therethrough. An outer vessel can be used to collect liquid that passes through the outlet, e.g., following centrifugation. Other embodiments of a device or kit as described herein include a flow path with one or more features (e.g., sharp features) that are configured to lyse a biological particle (e.g., a cell or particulate component thereof, e.g., nucleus) to separate or purify biological particles or the contents thereof. As a result of the purification, the invention can provide purified populations of biological particles (e.g., cells or particulate components thereof, e.g., organelles, such as nuclei).

The devices, kits, and methods described herein provide various advantages over other available devices and techniques known in the art. In particular, the devices, kits, and methods described herein may remove background (e.g., cytoplasmic) RNA and DNA, remove dead cells (e.g., provide samples with high cell viability), remove cellular debris, remove clumps and cell doublets, and remove poor quality nuclei. In one embodiment, the devices, kits, and methods improve the quality of a preparation of biological particles, e.g., cells or nuclei, wherein higher quality preparation generally minimize the presence of debris from lysed cells or non-intact nuclei. For example, quality can be assessed by cell viability measurement protocols. Viability can be assessed by staining (e.g., with Trypan blue) of a preparation followed by counting (e.g., automated counter or hemocytometer) of biological particles. Viability can be determined by measuring non-lysed cells that exclude staining, e.g., Trypan blue, while nuclei can be measured as not excluding staining. Alternatively, nuclei can be measured by fluorescent dyes, e.g., ethidium homodimer-1, may be used to measure and distinguish nuclei from debris. In addition, high quality nuclei can be measured by visualization with microscopy. High quality nuclei preparations are characterized by nuclei that are clump-free and debris-free nuclei, and/or have intact membranes that are smooth and round. Poor quality nuclei preparations are characterized by nuclei having a compromised membrane, e.g., ruffled in appearance. A compromised membrane may also comprise blebbing, which indicates a loss in nuclear membrane integrity which can result from over-lysis. In one embodiment, a high-quality nuclei preparation after cell lysis is demonstrated by measuring about less than 1% to about less than 10% live input cells. In other embodiments, a high-quality nuclei preparation after cell lysis is demonstrated by measuring about less than 1%, about less than 2%, about less than 3%, about less than 4%, about less than 5%, about less than 6% to about less than 7% live input cells, about less than 8%, about less than 9%, or about less than 10% live input cells.

By gently separation, purifying, and dispensing or storing the biological particles, the device or kit does not activate, deactivate, or change gene expression, viability, or functionality of the biological particles (e.g., cells or particulate components thereof, e.g., nuclei). This feature is particularly important when working with fixed or frozen cells or organelles (e.g., nuclei). Therefore, the samples being analyzed are not tainted by biological particles with altered expression patterns or alternative cellular subtypes. Other techniques are harsh, which reduces the efficiency of sample preparation. The devices and kits described herein reduce loss and provide enhanced purification, thereby providing a higher yield and increased purity of biological particles (e.g., cells or particulate components thereof, e.g., nuclei) for use in subsequent steps.

Devices and Kits

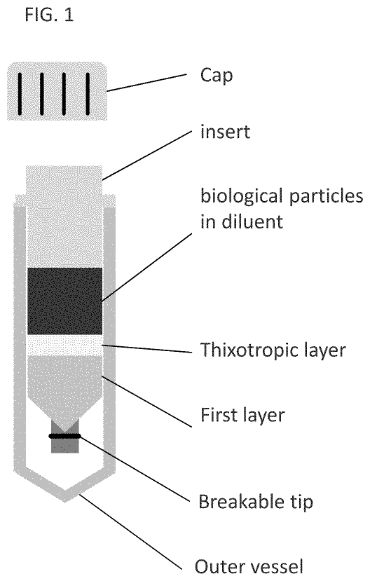

A kit for purifying biological particles (e.g., cells or particulate components thereof, e.g., organelles, such as nuclei) includes a vessel with an inlet to which a liquid can be provided. The vessel further includes a thixotropic layer disposed in the vessel (see FIG. 1). The vessel may further include an outlet to allow liquid to flow out of the vessel, e.g., to an outer vessel. The outer vessel can be used to collect liquid that passes through the outlet, e.g., following centrifugation.

Vessel

The devices and kits described herein may include a vessel. The vessel can be a removable insert that fits within an outer vessel. The vessel may contain an outlet. For example, the vessel may contain a removable or breakable tip such that liquid can flow through the outlet and once the tip is removed. The vessel may be any suitable geometry, such as cylindrical, conical, and the like. The vessel may be, for example, a microcentrifuge tube or a PCR tube or a similar device that can be removably inserted within a microcentrifuge tube or a PCR tube. The vessel may have a volume of e.g., 1 nL-100 mL (e.g., 1 nL, 2 nL, 3 nL, 4 nL, 5 nL, 6 nL, 7 nL, 8 nL, 9 nL, 10 nL, e.g., 10 nL-100 nL, e.g., 20 nL, 30 nL, 40 nL, 50 nL, 60 nL, 70 nL, 80 nL, 90 nL, 100 nL, e.g., 100 nL-1 μL, e.g., 200 nL, 300 nL, 400 nL, 500 nL, 600 nL, 700 nL, 800 nL, 900 nL, 1 μL, e.g., 1-10 μL, e.g., 2 μL, 3 μL, 4 μL, 5 μL, 6 μL, 7 μL, 8 μL, 9 μL, 10 μL, e.g., 10-100 μL, e.g., 20 μL, 30 μL, 40 μL, 50 μL, 60 μL, 70 μL, 80 μL, 90 μL, 100 μL, e.g., 100 μL-1 mL, e.g., 200 μL, 300 μL, 400 μL, 500 μL, 600 μL, 700 μL, 800 μL, 900 μL, 1 mL, e.g., 1 mL-10 mL, e.g., 2 mL, 3 mL, 4 mL, 5 mL, 6 mL, 7 mL, 8 mL, 9 mL, 10 mL, e.g., 10 mL-100 mL, e.g., 20 mL, 30 mL, 40 mL, 50 mL, 60 mL, 70 mL, 80 mL, 90 mL, or 100 mL). The vessel may have a volume of less than about 10,000 μL (e.g., less than about 1,000 μL, less than about 100 μL, or less than about 10 μL, e.g., from about 1 μL to about 10,000 μL, from about 10 μL to about 10,000 μL, from about 100 μL, to about 10,000 μL, from about 1,000 μL to about 10,000 μL, from about 10 μL to about 5,000 μL, from about 10 to about 1,000 μL, from about 100 μL to about 1,000 μL, from about 500 μL to about 1,000 μL, from about 1 μL to about 1,000 μL, from about 1 μL to about 500 μL, from about 1 μL to about 100 μL, from about 1 μL to about 50 μL, or from about 1 μL to about 10 μL).

The vessel may have a thickness of from about 10 μm to about 10 mm (e.g., from about 10 μm to about 1 mm, from about 10 μm to about 100 μm, from about 50 μm to about 100 μm, from about 100 μm to about 10 mm, from about 1 mm to about 10 mm, from about 500 μm to about 1 mm, from about 1 mm to about 5 mm, from about 1 mm to about 2 mm, e.g., about 1.5 mm). In some embodiments, the vessel may have thickness of from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1000 μm, e.g., about 200 μm, 300 μm, 400 μm, 500 μm, 600 μm, 700 μm, 800 μm, 900 μm, or 1000 μm, e.g., from about 1 mm to about 10 mm, e.g., 2 mm, 3 mm, 4 mm, 5 mm, 6 mm, 7 mm, 8 mm, 9 mm, or 10 mm.

The vessel may contain a cap that covers and/or seals the vessel. The cap can be attached to the vessel or a separate component. The cap can fit on the inlet and cover the vessel during subsequent use, e.g., during centrifugation.

In some embodiments, the vessel contains a thixotropic layer disposed within the vessel. The thixotropic layer separate layers of liquid with different densities to allow separation and purification of biological particles. The thixotropic layer may include a polymeric gel, such as a polyester. The thixotropic layer may be disposed within the vessel to allow for effective separation of liquids with different densities. For example, the thixotropic layer may have a disc or cylindrical shape within the vessel. The length, width, and height of the thixotropic layer may be at least, independently, e.g., 0.1 μm-10 mm (e.g., 0.1-1 μm, e.g., 0.1 μm, 0.2 μm, 0.3 μm, 0.4 μm, 0.5 μm, 0.6 μm, 0.7 μm, 0.8 μm, 0.9 μm, 1 μm, e.g., 1-10 μm, e.g., 1 μm, 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm). The thixotropic layer may have a volume of e.g., 1 nL-10 mL (e.g., 1 nL, 2 nL, 3 nL, 4 nL, 5 nL, 6 nL, 7 nL, 8 nL, 9 nL, 10 nL, e.g., 10 nL-100 nL, e.g., 20 nL, 30 nL, 40 nL, 50 nL, 60 nL, 70 nL, 80 nL, 90 nL, 100 nL, e.g., 100 nL-1 μL, e.g., 200 nL, 300 nL, 400 nL, 500 nL, 600 nL, 700 nL, 800 nL, 900 nL, 1 μL, e.g., 1-10 μL, e.g., 2 μL, 3 μL, 4 μL, 5 μL, 6 μL, 7 μL, 8 μL, 9 μL, 10 μL, e.g., 10-100 μL, e.g., 20 μL, 30 μL, 40 μL, 50 μL, 60 μL, 70 μL, 80 μL, 90 μL, 100 μL, e.g., 100 μL-1 mL, e.g., 200 μL, 300 μL, 400 μL, 500 μL, 600 μL, 700 μL, 800 μL, 900 μL, 1 mL, e.g., 1 mL-10 mL, e.g., 2 mL, 3 mL, 4 mL, 5 mL, 6 mL, 7 mL, 8 mL, 9 mL, 10 mL).

The thixotropic layer may separate a first layer of liquid having a first density and a second layer of liquid having a second density, thereby forming a density gradient within the vessel. The second liquid may have a density that is lower than the density of the first liquid.

The vessel may be loaded with a first layer of liquid having a first density. The first layer may be disposed below the thixotropic layer. The first layer may be positioned between the outlet and the thixotropic layer. The first layer may include iodixanol. For example, the first layer may include from about 10% to about 50% iodixanol (e.g., from about 15% to about 45%, from about 20% to about 40%, from about 25% to about 35%, from about 26% to about 34%, from about 27% to about 33%, from about 28% to about 32%, from about 29% to about 31%, from about 29.5% to about 30.5%, e.g., about 30%.

In another embodiment, a device may include a centrifuge tube containing a liquid having a predetermined density that is composed of tissue culture medium. For example, the centrifuge tube may contain a liquid having tissue culture medium and a concentration of from about 10% to about 50% iodixanol (e.g., from about 15% to about 45%, from about 20% to about 35%, from about 22% to about 32%, or from about 24% to about 30%).

The centrifuge tube may be, for example, a microcentrifuge tube or a PCR tube, or any other suitable structure configured to fit within a centrifuge.

In some embodiments, the density of the liquid is from about 1.11 g/mL to about 1.12 g/mL (e.g., from about 1.146 g/mL to about 1.175, e.g., about 1.1107 g/mL, 1.117 g/mL, 1.127 g/mL, 1.136 g/mL, 1.146 g/mL, 1.156 g/mL, 1.165 g/mL, or 1.175 g/mL. The liquid may contain iodixanol or a comparable component to create a similar density as iodixanol, such as ficoll, histopaque, or sucrose.

Advantages of using a liquid having tissue culture medium and the density described above allows easy removal of cellular debris, e.g., during centrifugation, while still maintaining viable conditions for the biological particles or particulate components thereof, e.g., nuclei.

Outer Vessel

In some embodiments, the invention employs an outer vessel that to house the vessel and optionally to collect a liquid from the outlet of the vessel, e.g., that is a removable insert within the outer vessel. The outer vessel may be any suitable geometry, such as cylindrical, conical, and the like. The outer vessel may be, for example, a microcentrifuge tube or a PCR tube or a similar device configured to house a microcentrifuge tube or a PCR tube. The outer vessel may have a volume may have a volume of e.g., 1 nL-100 mL (e.g., 1 nL, 2 nL, 3 nL, 4 nL, 5 nL, 6 nL, 7 nL, 8 nL, 9 nL, 10 nL, e.g., 10 nL-100 nL, e.g., 20 nL, 30 nL, 40 nL, 50 nL, 60 nL, 70 nL, 80 nL, 90 nL, 100 nL, e.g., 100 nL-1 μL, e.g., 200 nL, 300 nL, 400 nL, 500 nL, 600 nL, 700 nL, 800 nL, 900 nL, 1 μL, e.g., 1-10 μL, e.g., 2 μL, 3 μL, 4 μL, 5 μL, 6 μL, 7 μL, 8 μL, 9 μL, 10 μL, e.g., 10-100 μL, e.g., 20 μL, 30 μL, 40 μL, 50 μL, 60 μL, 70 μL, 80 μL, 90 μL, 100 μL, e.g., 100 μL-1 mL, e.g., 200 μL, 300 μL, 400 μL, 500 μL, 600 μL, 700 μL, 800 μL, 900 μL, 1 mL, e.g., 1 mL-10 mL, e.g., 2 mL, 3 mL, 4 mL, 5 mL, 6 mL, 7 mL, 8 mL, 9 mL, 10 mL, e.g., 10 mL-100 mL, e.g., 20 mL, 30 mL, 40 mL, 50 mL, 60 mL, 70 mL, 80 mL, 90 mL, or 100 mL). The outer vessel may have a volume of less than about 10,000 μL (e.g., less than about 1,000 μL, less than about 100 μL, or less than about 10 μL, e.g., from about 1 μL to about 10,000 μL, from about 10 μL to about 10,000 μL, from about 100 μL, to about 10,000 μL, from about 1,000 μL to about 10,000 μL, from about 10 μL to about 5,000 μL, from about 10 μL to about 1,000 μL, from about 100 μL to about 1,000 μL, from about 500 μL to about 1,000 μL, from about 1 μL to about 1,000 μL, from about 1 μL to about 500 μL, from about 1 μL to about 100 μL, from about 1 μL to about 50 μL, or from about 1 μL to about 10 μL). The outer vessel may have a volume that is the same or greater than the removable insert.

The outer vessel may have a thickness of from about 10 μm to about 10 mm (e.g., from about 10 μm to about 1 mm, from about 10 μm to about 100 μm, from about 50 μm to about 100 μm, from about 100 μm to about 10 mm, from about 1 mm to about 10 mm, from about 500 μm to about 1 mm, from about 1 mm to about 5 mm, from about 1 mm to about 2 mm, e.g., about 1.5 mm). In some embodiments, the vessel may have thickness of from about 10 μm to about 100 μm, e.g., about 10 μm, 20 μm, 30 μm, 40 μm, 50 μm, 60 μm, 70 μm, 80 μm, 90 μm, or 100 μm, e.g., from about 100 μm to about 1000 μm, e.g., about 200 μm, 300 μm, 400 μm, 500 μm, 600 μm, 700 μm, 800 μm, 900 μm, or 1000 μm, e.g., from about 1 mm to about 10 mm, e.g., 2 mm, 3 mm, 4 mm, 5 mm, 6 mm, 7 mm, 8 mm, 9 mm, or 10 mm.

The outer vessel may contain a cap that covers and/or seals the outer vessel. The cap can be attached to the outer vessel or may be a separate component. The cap can fit on the inlet of the outer vessel and cover the outer vessel after use, e.g., for storage of the components therein.

In some embodiments, the outer vessel may include a medium having a density of greater than about 1.0 g/m, e.g., to collect biological particles or the contents thereof. For example, the medium has a density from about 1.11 g/mL to about 1.12 g/mL (e.g., from about 1.146 g/mL to about 1.175, e.g., about 1.1107 g/mL, 1.117 g/mL, 1.127 g/mL, 1.136 g/mL, 1.146 g/mL, 1.156 g/mL, 1.165 g/mL, or 1.175 g/mL. The liquid may contain iodixanol or a comparable component to create a similar density as iodixanol, such as ficoll, histopaque, or sucrose.

Devices and Kits with Sharp Features or Filters

Another embodiment of a device as described herein for lysing, separating, or purifying biological particles (e.g., cells or particulate components thereof, e.g., nuclei) includes a flow path having an inlet, an outlet, and a filter and/or at least one feature with a corner radius of less than about 10 mm, e.g., less than about 1 mm, disposed in the flow path. For example, the sharp feature may have a corner radius of less than about 10 mm (e.g., less than about 9 mm, 8 mm, 7 mm, 6 mm, 5 mm, 4 mm, 3 mm, 2 mm, or 1 mm, e.g., less than about 990 μm, 980 μm, 970 μm, 960 μm, 950 μm, 940 μm, 930 μm, 920 μm, 910 μm, or 900 μm, e.g., less than about 800 μm, 700 μm, 600 μm, 500 μm, 400 μm, 300 μm, 200 μm, or 100 μm, e.g., less than about 90 μm, 80 μm, 70 μm, 60 μm, or 50 μm, e.g., less than about 45 μm, 40 μm, 35 μm, 30 μm, 25 μm, 20 μm, 15 μm, or 10 μm, e.g., less than about 9 μm, 8 μm, 7 μm, 6 μm, 5 μm, 4 μm, 3 μm, 2 μm or 1 μm, e.g., from about 10 mm to about 1 μm, from about 1 mm to about 1 μm, from about 500 μm to about 1 μm, from about 100 μm to about 1 μm, from about 10 μm to about 1 μm, from about 1 mm to about 10 μm, from about 100 μm to about 10 μm, from about 50 μm to about 10 μm, from about 10 μm to about 1 μm, from about 10 mm to about 1 mm, from about 10 mm to about 5 mm, from about 5 mm to about 500 μm, from about 1 mm to about 500 μm, or from about 1 mm to about 10 μm).

In some embodiments, the device includes at least two sharp features, e.g., each with a corner radius of less than about 10 mm, e.g., less than about 1 mm. For example, the device may have at least 2, 3, 4, 5, 6, 7, 8, 9, 10, 15, 20, 25, 30, 35, 40, 45, 50, 60, 70, 80, 90, 100, 200, 300, 400, 500, or more sharp features disposed within the flow path.

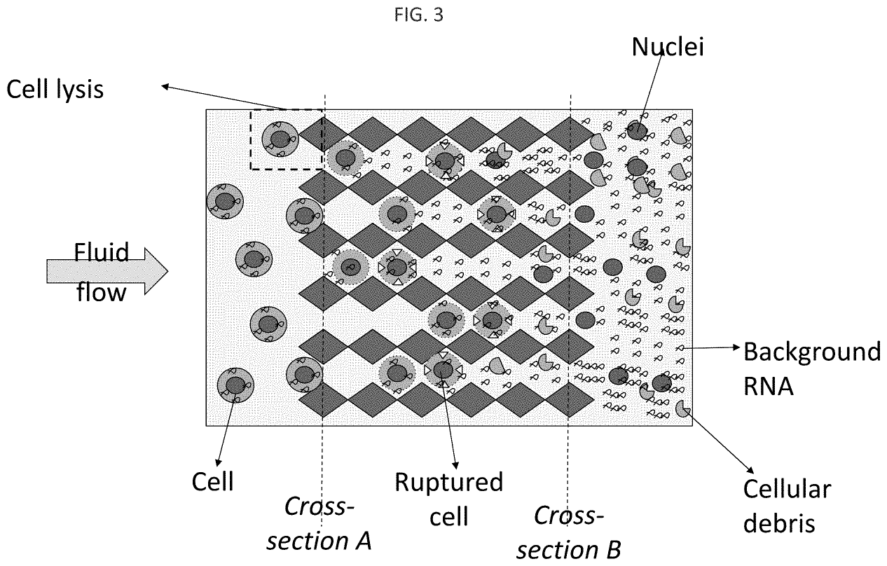

The sharp feature(s) disposed within the flow path allows the device to lyse a biological particle (e.g., a cell or particulate component thereof, e.g., an organelle, such as a nucleus) as it passes over the sharp feature (FIG. 3). The sharp feature may have a polygonal shape. For example, a cross-section of the feature may be a polygon with 3, 4, 5, 6, 7, 8, 9, 10, 11, 12, or more sides. The vertices of the polygon may have a corner radius of less than about 10 mm, e.g., less than about 1 mm. The device may include a protrusion with a plurality of sharp features or a plurality of protrusions, each having a sharp feature.

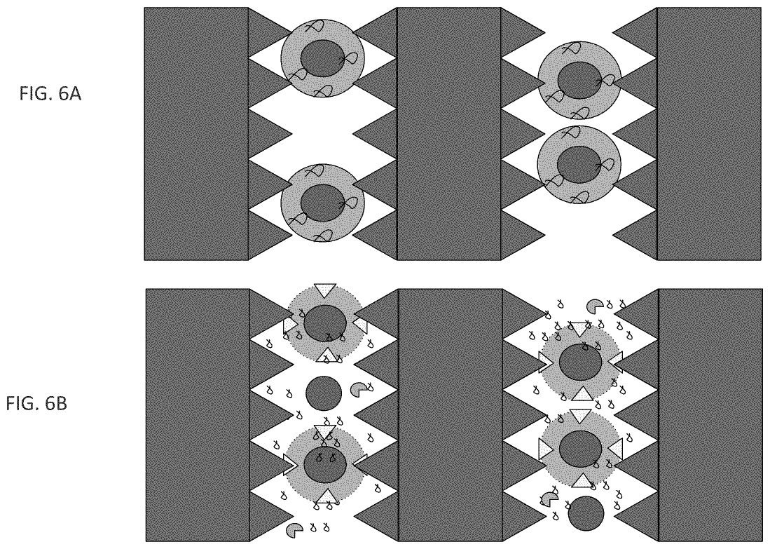

In some embodiments, the device includes a plurality of sharp features disposed in the flow path. The plurality of features may be arranged, for example, in a sawtooth pattern. In some embodiments, the features are positioned on opposite sides of the flow path (e.g., in register) in order to create narrow regions through which the biological particles pass through to increase the probability of being lysed (FIGS. 6A and 6B). In some embodiments, the device includes a plurality of matched pairs of features disposed in the flow path where each matched pair is in register.

The sharp features may be spaced apart, e.g., at predetermined intervals. For example, the sharp features (e.g., of a sawtooth pattern) may be spaced from about 1 μm to about 1 cm apart, e.g., from about 1 μm to 10 μm, e.g., 2 μm, 3 μm, 4 μm, 5 μm, 6 μm, 7 μm, 8 μm, 9 μm, or 10 μm, e.g., from about 10 μm to about 100 μm, e.g., 20 μm, 30 μm, 40 μm, 50 μm, 60 μm, 70 μm, 80 μm, 90 μm, or 100 μm, e.g., from about 100 μm to about 1 mm, e.g., 200 μm, 300 μm, 400 μm, 500 μm, 600 μm, 700 μm, 800 μm, 900 μm, or 1000 μm, e.g., 1 mm to about 10 mm, e.g., 2 mm, 3 mm, 4 mm, 5 mm, 6 mm, 7 mm, 8 mm, 9 mm, or 10 mm, e.g., from about 10 mm to about 100 mm, e.g., 20 mm, 30 mm, 40 mm, 50 mm, 60 mm, 70 mm, 80 mm, 90 mm, 100 mm, e.g., from about 100 mm to about 1 cm, e.g., 200 mm, 300 mm, 400 mm, 500 mm, 600 mm, 700 mm, 800 mm, 900 mm, or 1000 mm.

In some embodiments, the sharp features are arranged in a pattern, such as a linear pattern or a grid pattern (e.g., with regularly spaced intervals therebetween).

In some embodiments, the distance between the sharp features on opposite sides of the flow path are spaced apart, e.g., at predetermined intervals. The spacing may be configured to match a size of a biological particle to be lysed. For example, the distance between sharp features on opposite sides of the flow path may be less than the diameter of a cell or a particular component thereof, e.g., a nucleus, e.g., to allow for efficient lysis upon contact with the sharp features. In some embodiments, the sharp features are spaced on opposite sides of the flow path from about 1 μm to about 1 mm apart, e.g., from about 1 μm to 10 μm, e.g., 2 μm, 3 μm, 4 μm, 5 μm, 6 μm, 7 μm, 8 μm, 9 μm, or 10 μm, e.g., from about 10 μm to about 100 μm, e.g., 20 μm, 30 μm, 40 μm, 50 μm, 60 μm, 70 μm, 80 μm, 90 μm, or 100 μm, e.g., from about 100 μm to about 1 mm, e.g., 200 μm, 300 μm, 400 μm, 500 μm, 600 μm, 700 μm, 800 μm, 900 μm, or 1000 μm.

In some embodiments, the device includes a plurality of flow paths, each flow path having an inlet, and outlet, and at least one sharp features with a corner radius of less than about 10 mm, e.g., less than about 1 mm, disposed in the flow path. The plurality of flow paths may be substantially parallel (FIG. 3). The plurality of flow paths may each have a plurality of sharp features.

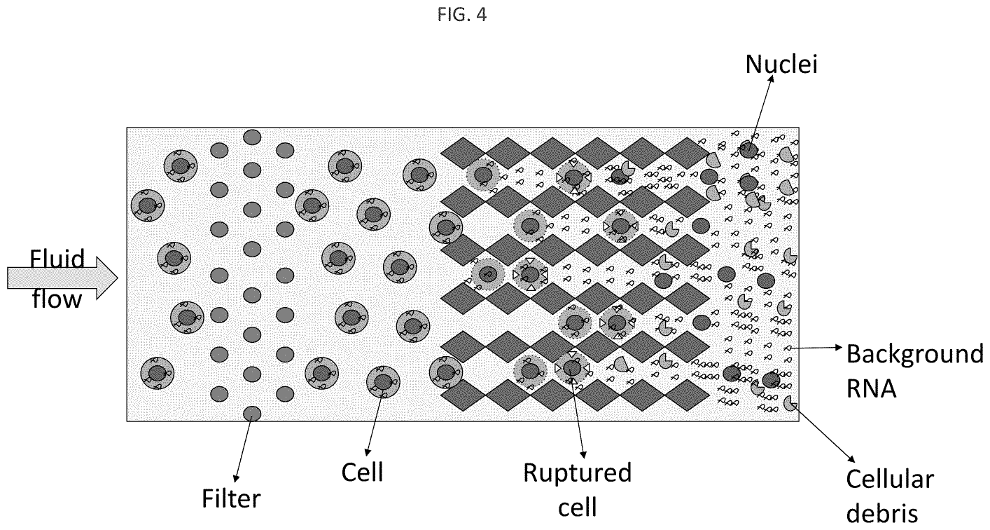

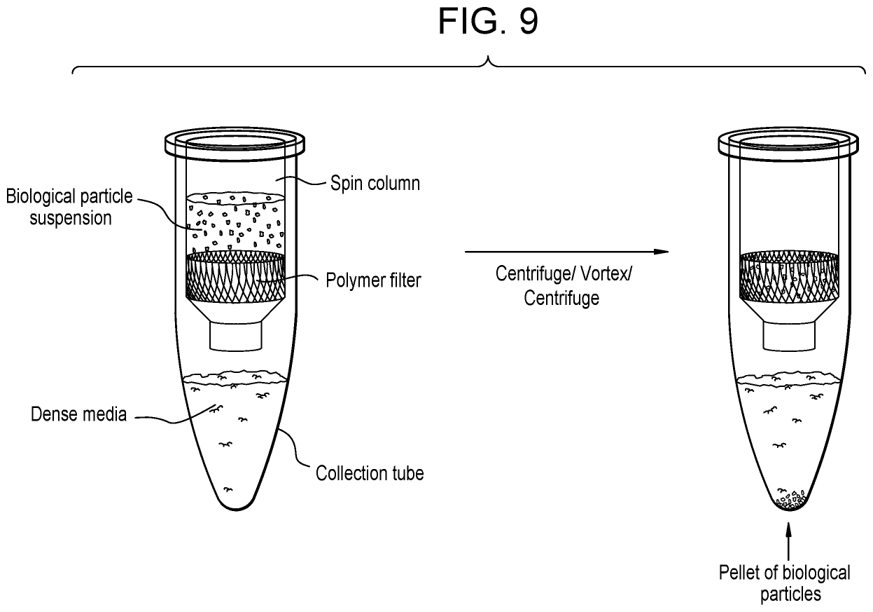

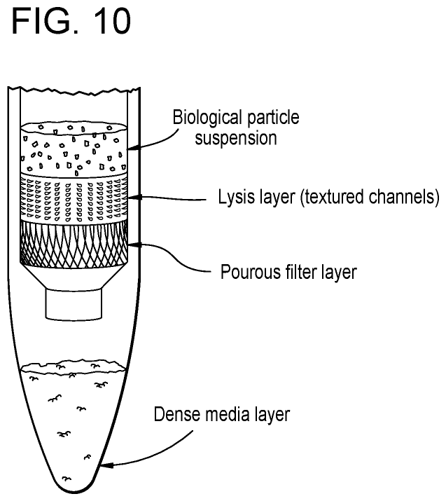

In some embodiments, the device includes a filter positioned in the flow path at or upstream of the outlet and configured to trap particles (e.g., biological particles) of a predetermined size (FIGS. 4 and 9). The filter may function to trap large biological particles (e.g., cells) or tissue particles, e.g., prior to coming into contact with sharp features. In one embodiment, the device includes a filter, e.g., as described herein, and a layer that includes a flow path having a sharp feature, e.g., with a corner radius of less than about 10 mm, e.g., less than about 1 mm, as described herein (FIG. 10). The layer may be disposed upstream or downstream of the filter. The layer may include a plurality of flow paths, e.g., in substantially parallel orientation. In some embodiments, the flow path includes a plurality of sharp features. In such an embodiment, the device may include a medium having a density of greater than about 1.0 g/m, e.g., to collect biological particles or the contents thereof.

The filter may include a polymer, e.g., a solid polymeric structure. For example, the filter may include polyethylene or polyethylene derivatives, cyclic olefin copolymers (COC), polymethylmethacrylate (PMMA), polydimethylsiloxane (PDMS), polycarbonate, polystyrene, polypropylene, polyvinyl chloride, polytetrafluoroethylene, polyoxymethylene, polyether ether ketone, polycarbonate, polystyrene, or the like.

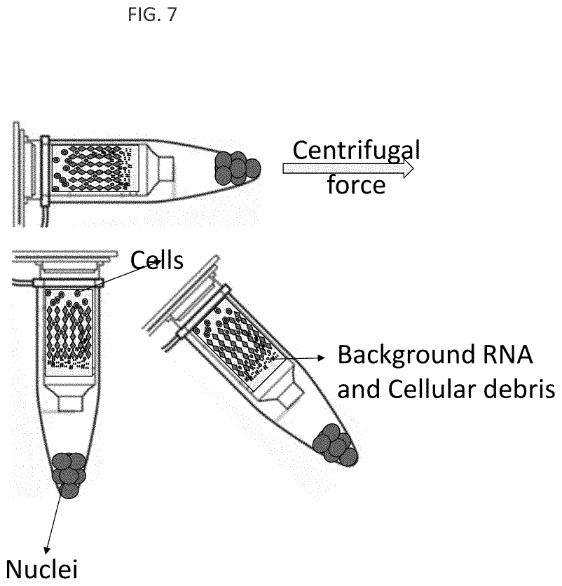

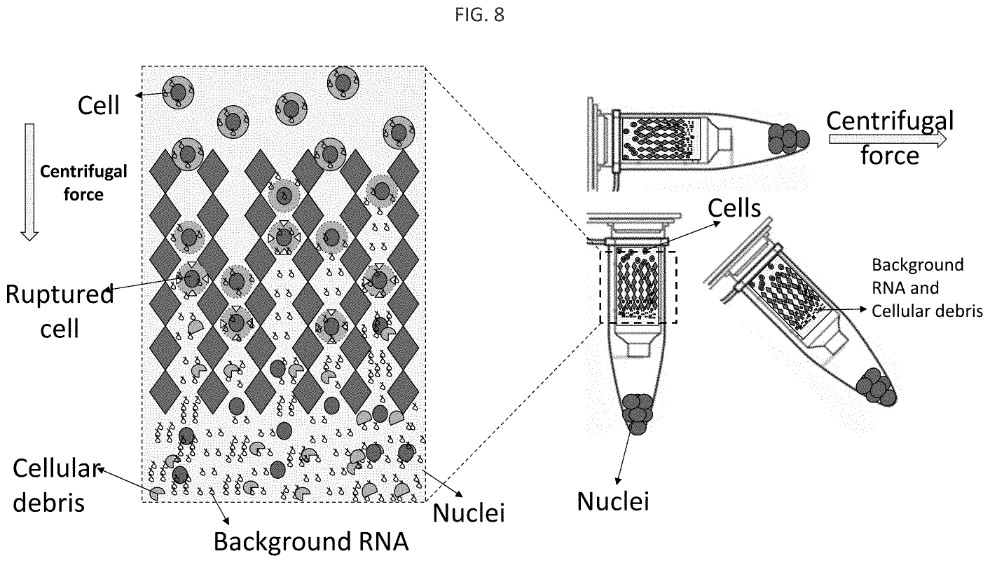

In some embodiments, the device with sharp feature(s) is employed in a kit in which the device is configured to fit within a vessel (e.g., an outer vessel) as described herein (see, e.g., FIGS. 7, 8 and 10). The vessel may be any suitable geometry, such as cylindrical, conical, and the like. The outer vessel may be, for example, a microcentrifuge tube or a PCR tube or a similar device. The vessel collects the liquid as it exits the outlet of the flow path. In some embodiments, the device is positioned in the outer vessel (e.g., microcentrifuge tube). The outer vessel may contain a liquid having a density that is greater than the density of the mixture that is provided to the device (e.g., greater than 1.0 g/m).

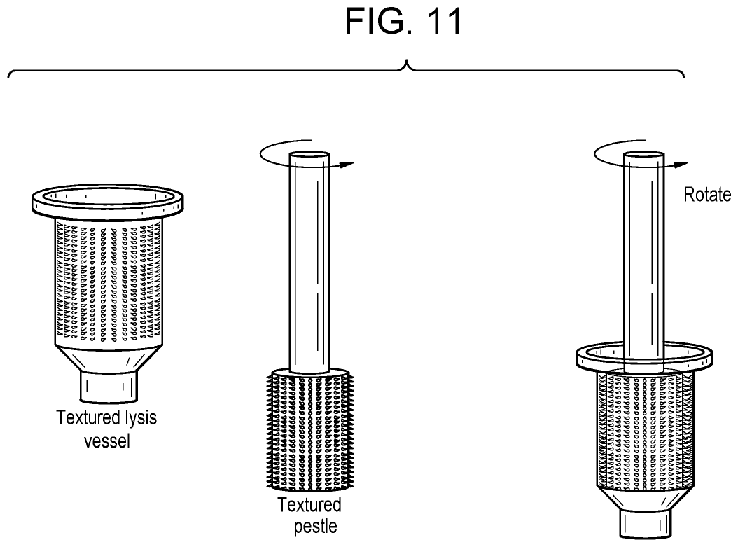

Also featured herein is a kit for lysing biological particles. The kit includes a vessel that includes at least one sharp feature (e.g., a plurality of features) having a corner radius of less than about 10 mm, e.g., less than about 1 mm, as described herein, disposed along a wall of the vessel. The kit further includes a pestle configured to fit within the vessel. The pestle may include at least one feature (e.g., a plurality of features) having a corner radius of less than about 1 mm disposed along a wall of the pestle. In some embodiments, the plurality of features of the vessel and/or the plurality of features of the pestle are arranged in a sawtooth pattern (FIG. 11). The pestle may contain a handle, e.g., for facile manipulation.

Sample

The samples that may be used with the devices and kits described herein may be any liquid, e.g., an aqueous liquid, that contains biological particles (e.g., cells or particulate components thereof, e.g., organelles, such as nuclei). The samples may contain insoluble substances, e.g., cellular debris, proteins, nucleic acids, etc. including extracellular molecules or analytes.

In some instances, unwanted molecules or analytes in a sample containing cells may be extracellular molecules or analytes. Extracellular analytes may be chemical, biological, or biochemical molecules or particles that are outside of a cell. The extracellular analytes may include any kind of molecules, such as nucleic acid molecules, peptides, proteins, substrates, a sequence of nucleic acids, a sequence of amino acids, or other kinds of molecules. Extracellular molecules may include extracellular nucleic acid molecules such as DNA and/or RNA or any other types of nucleic acid molecules and/or any combination thereof that are not inside a cell or cell nucleus. In some cases, extracellular molecules may be impurities in the sample. Additional disclosure regarding unwanted extracellular analytes is provided in U.S. Provisional Patent Application No. 63/109,972, which is incorporated here by reference in its entirety.

Extracellular molecules (e.g., extracellular nucleic acid molecules) may also be referred to as free-floating molecules (e.g., free-floating nucleic acid molecules), ambient molecules (e.g., ambient nucleic acid molecules), and/or background molecules (e.g., background nucleic acid molecules). Extracellular molecules may include molecules in a sample that are not inside a cell or cell nucleus which may act as impurities and may interfere with the quality of data obtained from analyzing the cells or cell nuclei of the sample. In some cases, extracellular molecules may be present without interfering with the quality of data obtained from analyzing the cells or cell nuclei of the sample.

In some instances, the extracellular molecules may include molecules such as extracellular peptides, proteins, substrates, a sequence of amino acids, chemicals, impurities and/or any combination thereof. For example, a sample with a cell or cell nucleus may further include extracellular molecules such as proteins and/or peptides.

In some instances, the extracellular molecules may include or be extracellular nucleic acid molecules. The extracellular nucleic acid molecules may include ribonucleic acid (RNA) or deoxyribonucleic acid (DNA). In some examples, extracellular nucleic acid may include at least one of messenger RNA (mRNA), chromosome, and genomic DNA (gDNA). In some examples, the extracellular nucleic acid molecules may have a size of at least about 5 base pairs (bp) or nucleotides (nt), for example, at least about 5 bp or nt, 10 bp or nt, 15 bp or nt, 20 bp or nt, 25 bp or nt, 30 bp or nt, 35 bp or nt, 40 bp or nt 50 bp or nt, 60 bp or nt, 70 bp or nt, 80 bp or nt, 90 bp or nt, 100 bp or nt, 200 bp or nt, 300 bp or nt, 400 bp or nt, 500 bp or nt, 600 bp or nt, 700 bp or nt, 800 bp or nt, 900 bp or nt, 1 kbp or knt, or larger in size. In some instances, extracellular nucleic acid molecules may be equal to or smaller than about: 500 bp or nt, 400 bp or nt, 300 bp or nt, 200 bp or nt, 100 bp or nt, 50 bp or nt, 40 bp or nt, 30 bp or nt, 20 bp or nt, 10 bp or nt, 5 bp or nt, or smaller, for example smaller than 50 bp or nt.

Extracellular analytes (e.g., extracellular nucleic acid molecules) may have been released in the sample from a cell or cell nucleus, for example, as a result of processing, treating, and/or manipulating a sample including a cell and/or a cell nucleus (e.g., during sample preparation). Such processing may have caused cell lysis or a loss of the integrity of the cell membrane and/or the nuclear membrane. This phenomenon may occur in any kind of cell, such as any cell type listed elsewhere herein or other types of cells. In some cases, a cell type that is more fragile may be more prone to getting lysed during sample preparation. Such cell type may be more likely to release extracellular molecules in the sample. Alternatively, the extracellular molecules (e.g., extracellular nucleic acid molecules) may have other origins and/or causes. Recognized is a need to address a contamination (or cross-contamination) of a sample with extracellular molecules (e.g., extracellular nucleic acid molecules) and their interference with data analysis (e.g., single cell analysis such as single cell sequencing or other sample processing and analysis techniques or procedures).

In some instances, the presence of extracellular molecules (e.g., extracellular nucleic acid molecules) in a sample including cells and/or cell nuclei may adversely affect and/or at least to some extent compromise the precision or quality of the results of the analysis (e.g., single cell analysis and/or data clustering results). For example, the goal may be to analyze the nucleic acid molecules in the cells and/or cell nuclei (e.g., intracellular nucleic acid molecules) of the sample. The method may further include clustering the data generated for the cells and/or the cell nuclei of the sample into more than one subpopulation (e.g., cluster the cell into multiple subpopulations). The method may further include identifying combinations of characteristics and/or parameters (e.g., markers) that may provide important information regarding each subpopulation and/or define the subpopulation in terms of a given state or condition of the sample or the subject, for example a disease marker or a diagnosis of the subject. The presence of extracellular molecules such as extracellular nucleic acid molecules may interfere with such clustering and/or identification in one or more ways. This phenomenon may also be referred to as cross-contamination. For example, the presence of extracellular molecules (e.g., ambient or background molecules such as nucleic acid molecules) may cause two or more subpopulations to blend together and the data (e.g., signal or sequencing reads) relating to a cell, cell nucleus, and/or the intracellular nucleic acid molecules thereof to be detected or categorized across two or more subpopulations. Extracellular molecules (e.g., extracellular nucleic acid molecules) may cause artifacts and/or noise in the data, alter the number of subpopulations resulted from the cluster analysis, interfere with the data in other ways, and/or any combinations thereof. This may cause imprecision in data and may adversely affect interpretation of results and/or decision making based on such data and/or data clustering. Therefore, depending on the application, there may be a need to ascertain the composition of a sample prior to use in, for example, single cell processing, including partitioning.

In some instances, the extracellular molecules (e.g., extracellular nucleic acid molecules), such as nucleic acid molecules inside a sample that are external to a cell or cell nucleus may generate information, such as signals (e.g., sequence reads) during sample processing and/or analysis. For example, a sample including a cell or cell nucleus which also includes extracellular nucleic acid molecules may be subjected to processing and analysis, for example, single cell sequencing (e.g., single cell RNA sequencing). In such case, the signals obtained from the extracellular nucleic acid molecules may be considered noise and may contaminate the data obtained from the intracellular nucleic acid molecules or data obtained from the nucleic acid molecules inside the cell nuclei. In this example, digesting or otherwise decreasing or removing the extracellular nucleic acid molecules from the sample (e.g., prior to sequencing) may enhance the quality of the single cell sequencing data and a clustering thereof.

In some instances, reduced amounts of extracellular molecules (e.g., extracellular nucleic acid molecules) or an absence thereof in the composition (e.g., processed sample) may result in more precise and/or more informative data with reduced noise, artifacts, imprecision, and/or error. Such data may include higher quality and may result in improved interpretation of results and/or more informed decision making. In some cases, the elimination of extracellular molecules (e.g., extracellular nucleic acid molecules) may reduce the time and expense of data analysis, for example, by providing cleaner data with reduced noise.

The devices described herein are particularly advantageous for small volumes of sample (e.g., less than 100 μL, 90 μL, 80 μL, 70 μL, 60 μL, 50 μL, 40 μL, 30 μL, 20 μL, 10 μL, 9 μL, 8 μL, 7 μL, 6 μL, 5 μL, 4 μL, 3 μL, 2 μL, or 1 μL), or volumes of samples that contain a low number of biological particles (e.g., a cell or particulate component thereof, e.g., a nucleus).

Biological particles in the samples may be purified using the invention before incorporation into droplets. Alternatively, the samples may be derived from droplets, e.g., following breaking or destabilization of droplets. Droplets generally refer to one liquid suspended in a second immiscible liquid and may be formed in which one or more biological particles are encapsulated within the droplet.

The biological particles may be suspended in any suitable buffer. For example, cells may be suspended in a suitable cell lysis buffer. The cell lysis buffer may include an RNAse inhibitor. The cell lysis buffer may include a surfactant (e.g., from about 0.01% to about 1.0% of the surfactant). In some embodiments, the cell lysis buffer includes 10 mM Tris-HCl (pH 7.4), 10 mM NaCl, 3 mM MgCl₂, 0.01% IGEPAL CA-630 (octylphenoxypolyethoxyethanol), 0.2 U/μl RNase Inhibitor (e.g., enzymatic RNAse inhibitor, e.g., Protector RNAse), 0.1% BSA, 2 mM spermine, and from about 0.01% to about 1.0% of a surfactant/detergent (e.g., TWEEN-20 (polysorbate 20) or IGEPAL CA-630 (octylphenoxypolyethoxyethanol). The lysis buffer may include, e.g., 10 mM Tris-HCl (pH 7.4), 10 mM NaCl, 3 mM MgCl₂, 0.01% IGEPAL CA-630 (octylphenoxypolyethoxyethanol), 0.2 U/μl SUPERase In RNase Inhibitor, 0.1% BSA, 2 mM spermine, and 0.1% TWEEN-20 (polysorbate 20). If organelles, such as nuclei, are to be purified from cells, the cells may be lysed in the lysis buffer. The nuclei may then be resuspended in an isotonic buffer for dispensing into the inlet of the vessel. The isotonic buffer may include an RNAse inhibitor. The isotonic buffer may include a surfactant (e.g., from about 0.01% to about 1.0% of the surfactant). A suitable isotonic buffer may include, e.g., 20 mM Tris-HCl (pH 7.4), 0.25 M sucrose, 25 mM KCl, 5 mM MgCl₂, 0.01% IGEPAL CA-630, 0.2 U/μl SUPERase In RNase Inhibitor, 0.1% BSA, 2 mM spermine, and 0.1% TWEEN-20.

The biological particles may be suspended or resuspended in a diluent. The diluent may be a second liquid having a second density, e.g., that is lower than the density of the first liquid. The second liquid may include iodixanol. For example, the second layer may include from about 5% to about 45% iodixanol (e.g., from about 10% to about 40%, from about 15% to about 35%, from about 20% to about 30%, from about 21 to about 29%, from about 22% to about 28%, from about 23% to about 27%, from about 24% to about 26%, from about 24.5% to about 26.5%, e.g., about 25%.

In some embodiments, the biological sample or the biological particles thereof (e.g., cells or nuclei) is obtained or provided by a device, kit, or method (e.g., lysis or purification method) as described herein. For example, the biological sample may be subject to multiple rounds of lysis and/or purification and used in different methods as described herein.

Droplet or Particle Source Devices

The devices and systems described herein may be used for purification of biological particles (e.g., cells or particulate components thereof, e.g., nuclei) for incorporation into droplets or particles. A device for producing droplets or particles may be used in conjunction with the kits and methods described herein. For example, one or more kits described herein may be used in conjunction with a device for producing droplets that contain the purified biological particles.

In general, droplets or particles are provided by a droplet or particle source. The droplets or particles may be first formed by flowing a first liquid through a channel and into a droplet or particle source region including a second liquid, i.e., the continuous phase, which may or may not be actively flowing.

Devices for producing droplets or particles include a droplet or particle source. The droplet or particle source provides droplets or particles, e.g., for subsequent use. The droplet or particle source may include a droplet or particle source region. Droplets or particles may be formed by any suitable method known in the art. In general, droplet formation includes two liquid phases. The two phases may be, for example, the sample phase and an oil phase. During formation, a plurality of discrete volume droplets or particles are formed.

The droplets may be formed by shaking or stirring a liquid to form individual droplets, creating a suspension or an emulsion containing individual droplets, or forming the droplets through pipetting techniques, e.g., with needles, or the like. The droplets may be formed made using a micro-, or nanofluidic droplet maker. Examples of such droplet makers include, e.g., a T-junction droplet maker, a Y-junction droplet maker, a channel-within-a-channel junction droplet maker, a cross (or “X”) junction droplet maker, a flow-focusing junction droplet maker, a micro-capillary droplet maker (e.g., co-flow or flow-focus), and a three-dimensional droplet maker. The droplets may be produced using a flow-focusing device, or with emulsification systems, such as homogenization, membrane emulsification, shear cell emulsification, and fluidic emulsification.

Discrete liquid droplets may be encapsulated by a carrier fluid that wets the microchannel. These droplets, sometimes known as plugs, form the dispersed phase in which the reactions occur. Systems that use plugs differ from segmented-flow injection analysis in that reagents in plugs do not come into contact with the microchannel. In T junctions, the disperse phase and the continuous phase are injected from two branches of the “T”. Droplets of the disperse phase are produced as a result of the shear force and interfacial tension at the fluid-fluid interface. The phase that has lower interfacial tension with the channel wall is the continuous phase. To generate droplets in a flow-focusing configuration, the continuous phase is injected through two outside channels and the disperse phase is injected through a central channel into a narrow orifice. Other geometric designs to create droplets would be known to one of skill in the art. Methods of producing droplets are disclosed in Song et al. Angew. Chem. 45: 7336-7356, 2006, Mazutis et al. Nat. Protoc. 8(5):870-891, 2013, U.S. Pat. No. 9,839,911; U.S. Pub. Nos. 2005/0172476, 2006/0163385, and 2007/0003442, PCT Pub. Nos. WO 2009/005680 and WO 2018/009766. In some embodiments, electric fields or acoustic waves may be used to produce droplets, e.g., as described in PCT Pub. No. WO 2018/009766.

In one embodiment, the droplet source region includes a shelf region that allows liquid to expand substantially in one dimension, e.g., perpendicular to the direction of flow. The width of the shelf region is greater than the width of the first channel at its distal end. In certain embodiments, the first channel is a channel distinct from a shelf region, e.g., the shelf region widens or widens at a steeper slope or curvature than the distal end of the first channel. In other embodiments, the first channel and shelf region are merged into a continuous flow path, e.g., one that widens linearly or non-linearly from its proximal end to its distal end; in these embodiments, the distal end of the first channel can be considered to be an arbitrary point along the merged first channel and shelf region. In another embodiment, the droplet source region includes a step region, which provides a spatial displacement and allows the liquid to expand in more than one dimension. The spatial displacement may be upward or downward or both relative to the channel. The choice of direction may be made based on the relative density of the dispersed and continuous phases, with an upward step employed when the dispersed phase is less dense than the continuous phase and a downward step employed when the dispersed phase is denser than the continuous phase. Droplet source regions may also include combinations of a shelf and a step region, e.g., with the shelf region disposed between the channel and the step region. Exemplary devices of this embodiment are described in WO 2019/040637, WO 2020/139844, and WO 2020/176882, the droplet forming devices of which are hereby incorporated by reference.

Biological Samples

The invention contemplates lysis and/or purification of biological particles from biological samples. Biological particles include, e.g., cells or particulate components thereof, e.g., organelles, such as a nucleus or a mitochondrion) and/or macromolecular constituents thereof (e.g., components of cells (e.g., intracellular or extracellular proteins, nucleic acids, glycans, or lipids) or products of cells (e.g., secretion products)). The biological particles may be provided by the devices, kits, and methods of the present invention.

Cellular analytes, or analytes originating from biological particles (e.g., cells or nuclei), may be used and may include, without limitation, any or all molecules or substances from or produced by a cell. Chemically, cellular analytes may include proteins, polypeptides, peptides, saccharides, polysaccharides, lipids, nucleic acids, combinations thereof and other biomolecules. A cellular analyte may include a protein, a metabolite, a metabolic byproduct, an antibody or antibody fragment, an enzyme, an antigen, a carbohydrate, a lipid, a macromolecule, or a combination thereof (e.g., proteoglycan) or other biomolecule. The cellular analyte may be a nucleic acid molecule. The cellular analyte may be a deoxyribonucleic acid (DNA) molecule or a ribonucleic acid (RNA) molecule. The DNA molecule may be a genomic DNA molecule. The cellular analyte may include coding or non-coding RNA. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA.

In some instances, the analytes may be contained in a sample. A sample may contain only analytes or may contain analytes in addition to one or more additional components. In some examples, the sample may contain biological particles. In some examples, biological particles may be cells, parts of cells or cell organelles, like a cell nucleus. A biological particle may include a cell, without any limitation on the kind or type of cell. Cells may be eukaryotic, prokaryotic or archaea. Cells may be eukaryotic cells from a cell line or cell culture sample. A cell may be a mammalian cell. A cell may be an animal cell. A cell may be a human cell. A cell may be from a cell culture. A cell may be from an immortalized cell line. A cell may be from a primary sample, such as a patient sample. A cell may be from a frozen stock of cells (e.g., cryopreserved cells). The cells may be adherent cells or suspension cells. The cells may be from a tissue sample, such as a biopsy, core biopsy, needle aspirate, or fine needle aspirate. The sample containing cells may come from bodily fluids, such as blood, urine or saliva. The sample may be a skin sample. The sample may be a cheek swab. The sample may contain peripheral blood mononuclear cells (PBMCs). Samples that include parts of or organelles from cells are also encompassed by this disclosure. In some examples, a sample may contain a cell nucleus from a eukaryotic cell.

Cells include, for example, a plant cell, animal cell, human cell, insect-derived cells, bacteria, algae, cardiomyocytes, stem cells, neurons, primary neurons, embryonic stem cells (ESCs), induced pluripotent stem cells (iPSCs), hepatocytes, primary heart valve cells, primary hematopoietic cells, gastrointestinal cells, lymphocytes, T-cells, B-cells, natural killer cells, dendritic cells, hematopoietic cells, beta cells, somatic cells, germ cells, embryos (human and animal), zygotes, gametes, and other types of cells.

A biological particle may be obtained by using lysis reagents in order to release the contents (e.g., contents of the cell or contents containing one or more analytes (e.g., bioanalytes)) of the biological particles. In such cases, the lysis agents can be contacted with the biological particle suspension concurrently with, or immediately prior to the introduction of the biological sample or biological particles. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be used with the biological particles (e.g., cells or particulate components thereof, e.g., nuclei) to cause the release of the biological particles' contents during purification. For example, in some cases, surfactant-based lysis solutions may be used to lyse cells, although these may be less desirable for emulsion-based systems where the surfactants can interfere with stable emulsions. In some cases, lysis solutions may include non-ionic surfactants such as, for example, TRITON X-100 and TWEEN 20. In some cases, lysis solutions may include ionic surfactants such as, for example, sarcosyl and sodium dodecyl sulfate (SDS). In some embodiments, lysis solutions are hypotonic, thereby lysing cells by osmotic shock. Electroporation, thermal, acoustic, or mechanical cellular disruption may also be used in certain cases.

In addition to the lysis agents, other reagents can also be used with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids.

Macromolecular components (e.g., bioanalytes) of individual biological particles (e.g., cells or particulate components thereof, e.g., nuclei) can be provided with unique identifiers (e.g., barcodes) such that upon characterization of those macromolecular components, at which point components from a heterogeneous population of cells may have been mixed and are interspersed or solubilized in a common liquid, any given component (e.g., bioanalyte) may be traced to the biological particle (e.g., cell) from which it was obtained. The ability to attribute characteristics to individual biological particles or groups of biological particles is provided by the assignment of unique identifiers specifically to an individual biological particle or groups of biological particles. Unique identifiers, for example, in the form of nucleic acid barcodes, can be assigned or associated with individual biological particles (e.g., cells or particulate components thereof, e.g., nuclei) or populations of biological particles (e.g., cells or particulate components thereof, e.g., nuclei), in order to tag or label the biological particle's macromolecular components (and as a result, its characteristics) with the unique identifiers. These unique identifiers can then be used to attribute the biological particle's components and characteristics to an individual biological particle or group of biological particles. In certain embodiments in which droplets are produced, this can be performed by forming droplets including the individual biological particle or groups of biological particles with the unique identifiers (via particles, e.g., beads), as described in the kits and methods herein.

In some aspects, the unique identifiers are provided in the form of oligonucleotides that comprise nucleic acid barcode sequences that may be attached to or otherwise associated with the nucleic acid contents of individual biological particle, or to other components of the biological particle, and particularly to fragments of those nucleic acids. In embodiments, the oligonucleotides are partitioned such that as between oligonucleotides in a given droplet, the nucleic acid barcode sequences contained therein are the same, but as between different droplets, the oligonucleotides can, and do have differing barcode sequences, or at least represent a large number of different barcode sequences across all of the droplets in a given analysis. In some aspects, only one nucleic acid barcode sequence can be associated with a given droplet, although in some cases, two or more different barcode sequences may be present.

The nucleic acid barcode sequences can include from 6 to about 20 or more nucleotides within the sequence of the oligonucleotides. In some cases, the length of a barcode sequence may be 6, 7, 8, 9, 10, 11, 12, 13, 14, 15, 16, 17, 18, 19, 20 nucleotides or longer. In some cases, the length of a barcode sequence may be at least 6, 7, 8, 9, 10, 11, 12, 13, 14, 15, 16, 17, 18, 19, 20 nucleotides or longer. In some cases, the length of a barcode sequence may be at most 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be 4, 5, 6, 7, 8, 9, 10, 11, 12, 13, 14, 15, 16 nucleotides or longer. In some cases, the barcode subsequence may be at least 4, 5, 6, 7, 8, 9, 10, 11, 12, 13, 14, 15, 16 nucleotides or longer. In some cases, the barcode subsequence may be at most 4, 5, 6, 7, 8, 9, 10, 11, 12, 13, 14, 15, 16 nucleotides or shorter.

Analyte moieties (e.g., oligonucleotides) can also include other functional sequences useful in processing of nucleic acids from biological particles contained in the sample. These sequences include, for example, targeted or random/universal amplification primer sequences for amplifying the genomic DNA from the individual biological particles within the droplets while attaching the associated barcode sequences, sequencing primers or primer recognition sites, hybridization or probing sequences, e.g., for identification of presence of the sequences or for pulling down barcoded nucleic acids, or any of a number of other potential functional sequences.

In an example, particles (e.g., beads) are provided that each include large numbers of the above-described barcoded oligonucleotides releasably attached to the beads, where all of the oligonucleotides attached to a particular bead will include the same nucleic acid barcode sequence, but where a large number of diverse barcode sequences are represented across the population of beads used. In some embodiments, hydrogel beads, e.g., beads having polyacrylamide polymer matrices, are used as a solid support and delivery vehicle for the oligonucleotides, e.g., into droplets, as they are capable of carrying large numbers of oligonucleotide molecules and may be configured to release those oligonucleotides upon exposure to a particular stimulus, as described elsewhere herein. In some cases, the population of beads will provide a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of oligonucleotide molecules attached. In particular, the number of molecules of oligonucleotides including the barcode sequence on an individual bead can be at least about 1,000 oligonucleotide molecules, at least about 5,000 oligonucleotide molecules, at least about 10,000 oligonucleotide molecules, at least about 50,000 oligonucleotide molecules, at least about 100,000 oligonucleotide molecules, at least about 500,000 oligonucleotides, at least about 1,000,000 oligonucleotide molecules, at least about 5,000,000 oligonucleotide molecules, at least about 10,000,000 oligonucleotide molecules, at least about 50,000,000 oligonucleotide molecules, at least about 100,000,000 oligonucleotide molecules, and in some cases at least about 1 billion oligonucleotide molecules, or more.

Moreover, a population of beads can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, the population can include at least about 1,000 oligonucleotide molecules, at least about 5,000 oligonucleotide molecules, at least about 10,000 oligonucleotide molecules, at least about 50,000 oligonucleotide molecules, at least about 100,000 oligonucleotide molecules, at least about 500,000 oligonucleotides, at least about 1,000,000 oligonucleotide molecules, at least about 5,000,000 oligonucleotide molecules, at least about 10,000,000 oligonucleotide molecules, at least about 50,000,000 oligonucleotide molecules, at least about 100,000,000 oligonucleotide molecules, and in some cases at least about 1 billion oligonucleotide molecules.

In some cases, it may be desirable to incorporate multiple different barcodes within a sample, either attached to a single or multiple particles, e.g., beads, within the sample. For example, in some cases, mixed, but known barcode sequences set may provide greater assurance of identification in the subsequent processing, for example, by providing a stronger address or attribution of the barcodes to a given sample or portion thereof, as a duplicate or independent confirmation of the output from a given sample.

Oligonucleotides may be releasable from the particles (e.g., beads) upon the application of a particular stimulus. In some cases, the stimulus may be a photo-stimulus, e.g., through cleavage of a photo-labile linkage that releases the oligonucleotides. In other cases, a thermal stimulus may be used, where increase in temperature of the particle, e.g., bead, environment will result in cleavage of a linkage or other release of the oligonucleotides form the particles, e.g., beads. In still other cases, a chemical stimulus is used that cleaves a linkage of the oligonucleotides to the beads, or otherwise results in release of the oligonucleotides from the particles, e.g., beads. In one case, such compositions include the polyacrylamide matrices described above for encapsulation of biological particles and may be degraded for release of the attached oligonucleotides through exposure to a reducing agent, such as dithiothreitol (DTT).

The samples described herein may contain either one or more biological particles (e.g., cells or particulate components thereof, e.g., nuclei), either one or more barcode carrying particles, e.g., beads, or both at least a biological particle and at least a barcode carrying particle, e.g., bead.

Methods of Purification

The invention provides methods for purifying biological particles (e.g., cells or particulate components thereof, e.g., organelles, such as nuclei), e.g., from a biological sample (e.g., a tissue sample, e.g., a frozen tissue sample). The biological sample may be lysed, e.g., via a method as described herein, via a lysis buffer, sonication, mechanical lysis, or a combination thereof. The methods described herein may be used to purify the biological particles, e.g., by at least 50% (e.g., by at least 55%, 60%, 70%, 75%, 80%, 85%, 90%, 95%, 99%, or more) relative to the starting mixture.

Biological particles are in general purified by providing a kit of the invention and providing a liquid mixture that includes biological particles to the inlet of the vessel. The density of the liquid mixture is less than the density of the liquid layer in the vessel. The thixotropic layer maintains the density gradient of the first liquid between the outlet and the thixotropic layer. The method typically includes centrifuging the vessel. The thixotropic layer separates the first layer and the liquid mixture and allows passage of the biological particles through the thixotropic layer to the first layer during centrifugation. Various cellular debris and full cells may remain in the top layer above the thixotropic layer, whereas particulate components, such as organelles (e.g., nuclei), may pellet at the bottom of the vessel.

The method may further include removing a supernatant liquid after centrifugation, e.g., via decanting or discarding the supernatant. The vessel may be inserted in an outer vessel to collect a flow through liquid. The method may include opening a tip of the vessel to create the outlet, such that liquid can flow through the outlet. The method may further include eluting the biological particles. Eluting may include centrifuging the vessel such that the biological particles exit the vessel via the outlet and are collected in the outer vessel. The method may further include washing the biological particles. The method may further include resuspending the biological particles, e.g., in a suitable storage buffer, such as an isotonic buffer, e.g., before or after eluting.

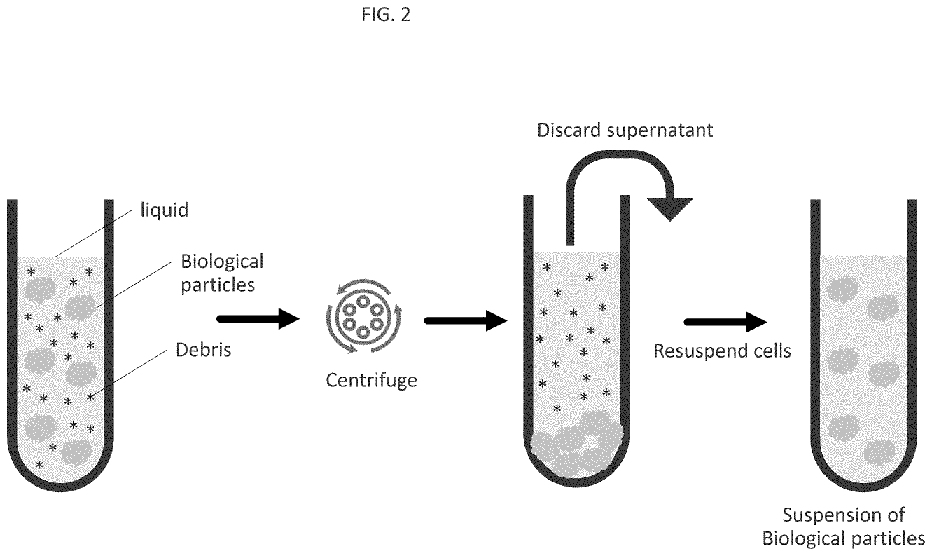

In another embodiment, the invention features a method of purifying biological particles with a centrifuge tube. The centrifuge tube contains a liquid composed of cell culture medium and a predetermined density of, e.g., from about 10% to about 50% iodixanol, e.g., from about 15% to about 45%, from about 20% to about 35%, from about 22% to about 32%, or from about 24% to about 30%, e.g., from about 24% to about 30% iodixanol, or a density of, e.g., from about 1.11 g/mL to about 1.2 g/mL (e.g., from about 1.146 g/mL to about 1.175, e.g., about 1.1107 g/mL, 1.117 g/mL, 1.127 g/mL, 1.136 g/mL, 1.146 g/mL, 1.156 g/mL, 1.165 g/mL, or 1.175 g/mL, e.g., from about 1.146 g/mL to about 1.175. The centrifuge tube may be centrifuged with the liquid having a suspension of biological particles (e.g., cells or nuclei). The density of the liquid allows the biological particles to pellet at the bottom of the centrifuge tube, and the debris remains in solution due to the small size (FIG. 2).

Centrifugation can be performed at a single speed or at multiple speeds. For example, a liquid mixture may be centrifuged at a first speed (e.g., from about 150 g to about 300 g, e.g., for about 5 to about 10 minutes) and then centrifuged at a second speed (e.g., higher than the first, e.g., from about 500 g to about 1000 g, e.g., for about 5 to about 10 minutes, e.g., about 10 minutes). By using two speeds of centrifugation, biological material may be pelleted at the first speed, the supernatant can be exchanged, and desired biological particles then pelleted at the bottom of the centrifuge tube at the second speed.

The method may further include removing a supernatant liquid after centrifugation, e.g., via decanting or discarding the supernatant. The method may further include washing the biological particles. The method may further include resuspending the biological particles, e.g., in a suitable storage buffer, such as an isotonic buffer, e.g., before or after eluting.

Suitable storage buffers include, for example, phosphate buffered saline, or a buffer containing, e.g., 20 mM Tris-HCl (pH 7.4), 0.25 M sucrose, 25 mM KCl, 5 mM MgCl₂, 0.01% IGEPAL CA-630, 0.2 U/μl SUPERase In RNase Inhibitor, 0.1% BSA, 2 mM spermine, and 0.1% TWEEN-20 (polysorbate 20).

In another embodiment, a first centrifugation step is performed in which all contents of the sample are pelleted. Following the first centrifugation, the supernatant may be removed, e.g., via decanting or discarding. A cell culture medium may then be added to the pellet. The cell culture medium may have a predetermined density of, e.g., from about 10% to about 50% iodixanol, e.g., from about 15% to about 45%, from about 20% to about 35%, from about 22% to about 32%, or from about 24% to about 30%, e.g., from about 24% to about 30% iodixanol, or a density of, e.g., from about 1.11 g/mL to about 1.2 g/mL, e.g., from about 1.146 g/mL to about 1.175, e.g., about 1.1107 g/mL, 1.117 g/mL, 1.127 g/mL, 1.136 g/mL, 1.146 g/mL, 1.156 g/mL, 1.165 g/mL, or 1.175 g/mL, e.g., from about 1.146 g/mL to about 1.175 e.g., from about 1.146 g/mL to about 1.175. When this sample is centrifuged, only certain (e.g., desired) biological particles (e.g., cells or particulate components thereof, e.g., nuclei) remain in the pellet.

In another embodiment, the methods described herein include purifying a second subset of biological particles using a kit as described herein, e.g., containing a device with an inlet, an outlet, and a filter positioned in the device. The kit further includes a vessel in which the device fits. The method includes providing the kit, providing a liquid mixture with biological particles to the inlet of the device; and centrifuging the device in the vessel. A first subset of the biological particles is trapped by the filter, and a second subset of the biological particles exits the outlet and is collected in the vessel.

In some embodiments, the vessel includes a medium having a density of greater than 1.0 g/m, and the second subset of biological particles is collected in the medium, e.g., pelleted during centrifugation.

In some embodiments, the methods of purification described herein allow a user to produce a population of biological particles having desired characteristics. For example, in some embodiments, purification generates populations of biological particles that include a suitable fraction of usable (e.g., viable) cells or particulate components thereof, e.g., organelles, e.g., nuclei (e.g., from 50% to 100%, from 60% to 100%, from 70% to 100%, from 80% to 100%, from 90% to 100%, or from 95% to 100% of biological particles). In some embodiments, the purification generates at least 10% e.g., at least 20%, 30%, 40%, 50%, 60%, 70%, 80%, 85%, 90%, 95%, 97%, 99%, or 100%, of the biological particles are usable for a desired purpose.

The methods of purification may be performed in manner that does not disturb the integrity of the cell, alter the characteristics (e.g., gene expression), activate, deactivate, differentiate, or reduce viability of the cell. In some embodiments, after purification, the relative expression level of each gene varies by less than 50%, less than 40%, less than 30%, less than 20%, less than 10%, less than 5%, less than 2%, or less than 1%. In some embodiments, the relative expression level of each gene of the cell remains substantially constant. In some embodiments, the methods do not activate or deactivate the cell. For example, the purification may avoid mechanically disrupting a cell, which disruption would trigger a signaling cascade that activates or deactivates the cell (e.g., immune cell, such as a T cell or B cell). In some embodiments, the methods of purification do not damage or kill the biological particles (e.g., cells or particulate components thereof, e.g., nuclei), e.g., causing leakage of the contents therein. In some embodiments, the methods of purification do not trigger apoptosis or necrosis.

The methods described herein may be used to purify biological particles (e.g., cells or particulate components thereof, e.g., nuclei), e.g., to produce populations of biological particles of uniform and predictable sizes with high throughput. The methods may be employed to purify biological particles for subsequent use, such as incorporation into droplets as microscale chemical reactors, where the volumes of the chemical reactants are small (˜pLs).

Methods of Lysis

In another embodiment, the invention features a method of lysing biological particles with a device with one or more sharp features. The device includes a flow path having an inlet and an outlet and at least one sharp feature disposed in the flow path. The method includes providing the device and flowing a liquid mixture containing the biological particles through the flow path (e.g., via the inlet). The biological particles are lysed upon interaction with the sharp feature, and at least a portion of the biological particles or the contents thereof exit the flow path via the outlet. The method may further include washing the biological particles in the flow path, e.g., one or more times, e.g., with a suitable wash buffer (e.g., phosphate buffered saline).

In some embodiments, the method includes providing magnetic particles to the liquid mixture or to the flow path (FIG. 4). The magnetic particles (e.g., beads) may be configured to bind to and/or capture biological material, such as background (e.g., cytoplasmic) RNA after cell rupture. The magnetic particles may also increase the efficiency of lysis of the biological particles. The magnetic particles may be used to separate out the biological material attached to the magnetic particles (e.g., by pelleting the magnetic particles) or using a magnet to selectively remove or move the magnetic particles with the biological particles attached thereto.

In another embodiment, the invention features a method of lysing biological particles with a device with one or more sharp features that is employed with a vessel. The method includes placing the device in the vessel, e.g., a centrifuge tube. The method further includes providing a liquid mixture including the biological particles to the inlet of the device and centrifuging the vessel. Upon centrifugation, the at least one feature lyses the biological particles, and at least a portion of the biological particles or the contents thereof exit the flow path via the outlet and are collected in the vessel.

In some embodiments, desired biological particles (e.g., nuclei) are collected in the vessel. In some embodiments, undesired biological particles are collected in the vessel.

In some embodiments, different biological materials preferentially exit the outlet of the flow path at different times, thereby allowing separation. For example, in one embodiment, desired biological particles, such as nuclei, exit the outlet first and are collected in the vessel, while undesired biological materials, such as cellular debris, background (e.g., cytoplasmic) RNA, or other ruptured cellular components, remain in the flow path. In some embodiments, undesired biological materials, such as cellular debris, background (e.g., cytoplasmic) RNA, or other ruptured cellular components exit the outlet first and are collected in the vessel, while desired biological particles, such as nuclei, remain in the flow path. The desired particles can be washed one or more times to remove excess undesired particles.

In some embodiments, the methods of lysis described herein employ a device with a filter, e.g., positioned in the flow path at or upstream of the outlet and configured to trap particles of a predetermined size. The filter may function, e.g., as a size-based filter in the flow path or as a series of obstacles. The filter may trap desired biological particles. Alternatively, the filter may trap undesired biological material.

The filter may also be employed upstream of the sharp features, e.g., to remove large debris (FIGS. 4 and 10).

In some embodiments, the methods for lysis of biological particles include using a kit as described herein containing a device (e.g., having an inlet and an outlet and layer with a flow path having at least one feature with a corner radius of less than about 1 mm disposed in the flow path) and a vessel to house the device. The method includes providing the kit, providing a liquid mixture with biological particles to the inlet of the device; and centrifuging the device in the vessel. The at least one feature lyses the biological particles, and at least a portion of the contents of the biological particle exits the flow path via the outlet and is collected in the vessel. In some embodiments, the vessel includes a medium having a density of greater than 1.0 g/m, and the contents of the biological particles are collected in the medium, e.g., pelleted during centrifugation. In some embodiments, the device further includes a filter disposed in the device, and the filter traps biological particles of a predetermined size in the filter, e.g., prior to or following lysis.

In another embodiment, the methods of lysis include employing a kit with a vessel and a pestle, e.g., each having one or more features having a corner radius of less than about 1 mm disposed along a wall of the vessel and/or the pestle. The method includes providing the kit; providing a liquid mixture containing biological particles in the vessel; and rotating the pestle (e.g., one or more times) in the vessel. The features of the vessel and/or the pestle lyse the biological particles (FIG. 11).

In some embodiments, the methods of lysis described herein allow a user to perform lysis in the absence of lysis reagents, such as surfactants. Additionally, the methods of lysis may be performed in the presence of amounts of lysis agents that are too low to cause lysis on their own.

The methods of lysis may be performed in a manner that does not disturb the integrity of certain contents of the biological particle after lysis (e.g., a cellular component, such as a nucleus), alter the characteristics (e.g., gene expression), activate, deactivate, differentiate, or reduce viability of certain contents of the biological particle. In some embodiments, after lysis, the relative expression level of each gene varies by less than 50%, less than 40%, less than 30%, less than 20%, less than 10%, less than 5%, less than 2%, or less than 1% in a cellular component (e.g., a nucleus or mitochondrion). In some embodiments, the relative expression level of each gene of the cellular component remains substantially constant. In some embodiments, the methods do not activate or deactivate the biological particle. For example, the lysis may avoid mechanically disrupting a nucleus, which disruption would trigger a signaling cascade that activates or deactivates the nucleus. In some embodiments, the methods of lysis do not damage or kill the biological particles (e.g., nuclei), e.g., causing leakage of the contents therein.

Downstream Methods

The methods of lysis and/or purification as described herein may be used to produce biological particles for downstream methods or applications. A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Devices, systems, compositions, and methods of the present disclosure may be used for various applications, such as, for example, processing a single analyte (e.g., bioanalytes, e.g., RNA, DNA, or protein) or multiple analytes (e.g., bioanalytes, e.g., DNA and RNA, DNA and protein, RNA and protein, or RNA, DNA and protein) from a single cell or nucleus. For example, a biological particle (e.g., a cell, a nucleus, or virus) can be formed in a droplet, and one or more analytes (e.g., bioanalytes) from the biological particle (e.g., cell or nucleus) can be modified or detected (e.g., bound, labeled, or otherwise modified by an analyte moiety) for subsequent processing. The multiple analytes may be from the single cell or nucleus. This process may enable, for example, proteomic, transcriptomic, and/or genomic analysis of the cell/nucleus or population thereof (e.g., simultaneous proteomic, transcriptomic, and/or genomic analysis of the cell/nucleus or population thereof).

Methods of modifying analytes include providing a plurality of particles (e.g., beads) in a liquid carrier (e.g., an aqueous carrier); providing a sample containing an analyte (e.g., as part of a cell, a nucleus or component or product thereof) in a sample liquid; and using the device to combine the liquids and form an analyte droplet containing one or more particles and one or more analytes (e.g., as part of one or more cells, nuclei, or components or products thereof). Such sequestration of one or more particles with analyte (e.g., bioanalyte associated with a cell or a nucleus) in a droplet enables labeling of discrete portions of large, heterologous samples (e.g., single cells or nuclei within a heterologous population). Once labeled or otherwise modified, droplets or particles can be subsequently sorted or combined (e.g., by breaking an emulsion), and the resulting liquid can be analyzed to determine a variety of properties associated with each of numerous single cells or nuclei.

Another embodiment provides methods of single-cell (or single-nucleus) nucleic acid sequencing, in which a heterologous population of cells/nuclei can be characterized by their individual gene expression, e.g., relative to other cells/nuclei of the population. Methods of barcoding cells/nuclei discussed above and known in the art can be part of the methods of single-cell (or single nucleus) nucleic acid sequencing provided herein. After barcoding, nucleic acid transcripts that have been barcoded are sequenced, and sequences can be processed, analyzed, and stored according to known methods. In some embodiments, these methods enable the generation of a genome library containing gene expression data for any single cell or nucleus within a heterologous population.

In another aspect, the present disclosure provides an alternative method for removing or depleting unwanted components (e.g., dead cells or extracellular molecules) from a sample with biological particles. In one embodiment, the method includes providing a sample with biological particles and unwanted components originating from biological particles (e.g., live or dead cells). In an additional embodiment, the biological particles are live (or intact) cells or intact nuclei. In an additional embodiment, the biological particles are live (or intact) cells or intact nuclei. In another embodiment, the unwanted components include one or more of dead or dying cells, non-intact nuclei, extracellular analytes or molecules, and other debris. In one embodiment, the method includes magnetically labelling unwanted components (e.g., dead cells) and/or magnetically capturing or trapping unwanted components (e.g., extracellular molecules, such as background RNA) in the sample to provide a suspension (e.g., an aqueous suspension or liquid) with unwanted components that are magnetically labelled and biological particles (e.g., live cells or intact nuclei) that are un-labelled, i.e., not magnetically labelled. In the case of unwanted extracellular molecules, a magnetic particle configured to bind or trap such molecules may be used. In one embodiment, the method includes providing a device for separation that includes a flow path with an inlet and an outlet (and optionally, a vessel) and a magnetic source disposed to exert a magnetic field on the particles in the flow path or on the magnetic particles after they exit the flow path. In one embodiment, the method further includes flowing the suspension into the flow path, e.g., via the inlet. In a further embodiment, the device is subjected to conditions that allow the unwanted components that are magnetically labelled to be immobilized at a location in the sample, e.g., via a magnetic source.

Other embodiments are in the claims.

1-14. (canceled)

15. A kit comprising an outer vessel and a removable insert configured to fit in the vessel, the removable insert comprising:
(a) an inlet;
(b) a first layer comprising a first liquid having a first density; and
(c) a thixotropic layer disposed above the first layer, wherein the thixotropic layer is configured to separate the first layer and a diluent having a density lower than the first density provided to the inlet of the insert.

16. The kit of claim 15, wherein the removable insert comprises an outlet.

17. (canceled)

18. The kit of claim 15, wherein the first layer comprises iodixanol.

19. The kit of claim 18, wherein the first layer comprises about 30% iodixanol.

20. (canceled)

21. The kit of claim 15, wherein the diluent comprises about 25% iodixanol.

22-23. (canceled)

24. The kit of claim 15, further comprising a cell lysis buffer and/or an isotonic buffer.

25-27. (canceled)

28. The kit of claim 15, further comprising a cap configured to seal the inlet of the removable insert.

29-32. (canceled)

33. A device for lysing biological particles comprising a flow path comprising:
(a) an inlet;
(b) an outlet; and
(c) at least one feature with a corner radius of less than about 1 mm disposed in the flow path.

34. The device of claim 33, wherein the flow path comprises a plurality of features, each feature having a corner radius of less than about 1 mm and disposed in the flow path.

35. The device of claim 34, wherein at least two of the features are positioned in register and on opposite sides of the flow path.

36. The device of claim 34, wherein the plurality of features are arranged in a sawtooth pattern.

37. The device of claim 33, wherein the device comprises a plurality of flow paths, each flow path comprising:
(a) an inlet;
(b) an outlet; and
(c) at least one feature with a corner radius of less than about 1 mm disposed in the flow path.

38. The device of claim 37, wherein the plurality of flow paths are substantially parallel.

39. The device of claim 33, further comprising a filter positioned in the flow path at or upstream of the outlet and configured to trap particles of a predetermined size.

40. A kit comprising the device of claim 33 and a vessel, wherein the device is configured to fit in the vessel.

41-47. (canceled)

48. A method for lysing biological particles comprising:
(a) providing the device of claim 33; and
(b) flowing a liquid mixture comprising the biological particles through the flow path,
wherein the at least one feature lyses the biological particles, and wherein at least a portion of the biological particles or the contents thereof exit the flow path via the outlet.

49. (canceled)

50. The method of claim 48, further comprising providing magnetic particles that bind to at least a portion of the biological particles or the contents thereof.

51. (canceled)

52. A kit comprising a vessel and a device configured to fit in the vessel, the device comprising:
(a) an inlet;
(b) an outlet; and
(c) a filter positioned in the device at or upstream of the outlet and configured to trap particles of a predetermined size.

53. The kit of claim 52, wherein the device further comprises a layer disposed at or upstream of the outlet and comprising a flow path with at least one feature with a corner radius of less than about 1 mm and disposed in the flow path.

54. The kit of claim 53, wherein the layer comprises a plurality of flow paths, each flow path in the layer comprising:
(i) an inlet;
(ii) an outlet; and
(iii) at least one feature with a corner radius of less than about 1 mm disposed in the flow path.

55. The kit of claim 54, wherein the plurality of flow paths in the layer are substantially parallel.

56. (canceled)

57. A method for purifying a second subset of biological particles comprising:
(a) providing the kit of claim 52;
(b) providing a liquid mixture comprising the biological particles to the inlet of the device; and
(c) centrifuging the device in the vessel;
wherein a first subset of the biological particles is trapped in the filter and a second subset of the biological particles exits the outlet and is collected in the vessel.

58. The method of claim 57, wherein the vessel comprises a medium having a density of greater than 1.0 g/m, and the second subset of biological particles is collected in the medium.

59. A method for lysing biological particles comprising:
(a) providing the kit of claim 53;
(b) providing a liquid mixture comprising the biological particles to the inlet of the device; and
(c) centrifuging the device in the vessel;
wherein the at least one feature lyses the biological particles, and wherein at least a portion of the contents of the biological particles exits the flow path via the outlet and are collected in the vessel.

60-62. (canceled)

63. A method of purifying a biological particle comprising:
(a) providing the kit of claim 15;
(b) providing a liquid mixture comprising the biological particle to the inlet of the vessel, wherein the density of the liquid mixture is less than the first density; and
(c) centrifuging the vessel;
wherein the thixotropic layer separates the first layer and the liquid mixture and allows passage of the biological particle to the first layer during centrifugation.
